(19) United States
(12) Patent Application Publication
(10) Pub. No.: US 2025/0339404 A1
(43) Pub. Date: Nov. 6, 2025

(54) COMBINATION THERAPY USING A CD47-SIRP alpha BLOCKING AGENT AND AZACITIDINE

(71) Applicant: Aurigene Oncology Limited, Bangalore (IN)

(72) Inventors: Sandeep Sadashiv Patil, Sangli (IN); Muralidhara Ramachandra, Bangalore (IN); Akhil Kumar, Bangalore (IN); Manjunath Krishnappa, Bangalore (IN)

(21) Appl. No.: 19/198,975

(22) Filed: May 5, 2025

(30) Foreign Application Priority Data
May 3, 2024 (IN) IN202441035249

(51) Int. Cl. A61K 31/4245; A61K 9/00; A61K 31/706; A61P 35/02

(52) U.S. Cl. CPC A61K 31/4245; A61K 9/0053; A61K 31/706; A61P 35/02

(57) ABSTRACT

The present disclosure relates to a CD47-SIRPα blocking agent of formula (I) stereoisomers thereof, and pharmaceutically acceptable salts, solvates, amides and esters of the compound of formula (I) and stereoisomer thereof and Azacitidine for use in combination with Azacitidine in a method of treating or delaying progression of a disease or a disorder mediated by CD47-SIRPα pathway in a human subject suffering from said disease, such as hematological cancer.

### TECHNICAL FIELD

The present disclosure relates to a method of treating or delaying progression of a disease or a disorder mediated by CD47-SIRPα pathway in a human subject suffering from said disease. More particularly the disclosure relates to use in treatment of cancer, such as a haematological cancer.

### BACKGROUND

Cluster of differentiation 47 (CD47) is a key immune checkpoint for macrophage mediated phagocytosis. CD47 is an immunoglobulin that is overexpressed on the surface of many types of cancer cells, including hematologic cancers such as Acute myeloid leukaemia (AML) and myelodysplastic syndrome (MDS). AML is genetically heterogeneous hematologic malignancy characterized by multiple mutations and epigenetic dysregulation. While MDS is a heterogeneous group of myeloid disorders defined by a common set of features, of which the most prominent is morphologic bone marrow and peripheral blood dysplasia associated with inefficient haematopoiesis, the development of peripheral cytopenia and an increased risk of transformation to AML.

CD47 interacts with signalling complex with signal-regulatory protein α (SIRPα) to form CD47-SIRPα axis, which enables the escape of these cancer cells from macrophage-mediated phagocytosis and thus protects the cancer cells.

Targeting CD47-SIRPα axis using CD47-SIRPα blocking agents has emerged as one of the promising new immunotherapy approaches that target innate immune response. However, given the ubiquitous expression of CD47 on cancer cells as well as healthy cells, particularly hematopoietic cells, this approach has an inherent risk of safety, often resulting in cytotoxicity, more particularly anemia, thrombocytopenia, hyperbilirubinemia, leukopenia, and neutropenia. The use of CD47-SIRPα blocking agents may also affect solid tissues rich in macrophages such as liver, lung, and brain.

For solid tumors, there have been attempts to control or mitigate such side effects by using novel drug delivery systems for tumor specific delivery of CD47-SIRPα blocking agents, such as, tumor-targeting nanoparticles, or the use of antibody-drug conjugates for targeted delivery. For solid tumors as well as hematological cancers, monoclonal antibodies targeting CD47-SIRPα axis have also been tested in clinical setting (Zhao H, Song S S, Ma J, Yan Z Y, Xie H W, Feng Y and Che S S (2022) CD47 as a promising therapeutic target in oncology. Front. Immunol. 13:757480).

Although approaches that block the CD47-SIRPα signalling axis have yielded promising preclinical results, with prominent induction of phagocytosis in various cancer types, reports of successful (experimental) clinical use of small molecule CD47 signalling pathways inhibitors for treatment of cancer, in particular hematological cancer, are extremely scarce. Treatment with CD47-SIRPα blocking therapeutics in patients is invariably associated with a lack of therapeutic effect at the maximum tolerated dose (MTD) and significant dose-limiting toxicity (DLT), most notably due to anaemia and thrombocytopenia.

It is an object of the present invention to provide methods that are efficacious in treating or delaying the progression of a disease or a disorder mediated by CD47-SIRPα pathway, with an acceptable safety profile.

### SUMMARY

The present disclosure provides a safe and effective treatment of diseases or disorders mediated by CD47-SIRPα pathway, using a CD47-SIRPα blocking agent according to formula (I):

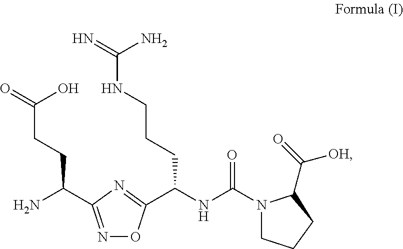

stereoisomers thereof, or a pharmaceutically acceptable salt, solvate, amide or ester of the compound of formula (I) or stereoisomer thereof, in combination with azacitidine or a pharmaceutically acceptable salt thereof.

More in particular, the present disclosure is based, in one part, on the surprising finding that the CD47-SIRPα blocking agent of formula (I), as depicted below, is safe and well-tolerated up to a dose of 400 mg per oral BID, or a total daily dose of 800 mg, with PD biomarker analysis indicating favourable modulation of cytokines and chemokines, which is indicative of the efficacy of the compound of formula (I) against cancer. Most surprisingly, the hematological safety of the compound of formula (I) was exceptionally high, with no grade 3 or grade 4 adverse events, and no hemolytic anemia in any of the tested subjects. The experimental findings underlying the invention, provide strong support for the use of the CD47-SIRPα blocking agent in combination with Azacitidine, in treating or delaying the progression of hematological cancers such as Acute Myeloid Leukemia (AML) or Myelodysplastic Syndrome (MDS) in human subjects.

The compound of formula (I) has been disclosed in PCT publication WO2019/138367, which is directed to a larger group of 1,2,4-oxadiazole compounds as small molecule CD47 signalling pathways inhibitors. WO2020/095256 discloses several 1,2,4-oxadiazole compounds for use in combination with one or more anticancer agents. Neither one of these documents teaches or suggests using the compound of formula (I) in combination with azacitidine, so as to provide a method for treating or delaying the progression of a disease or a disorder mediated by CD47-SIRPα pathway, which is efficacious and safe. In the field of cancer treatment in particular, selecting the most appropriate combination partners to provide a positive balance of efficacy and safety has been a challenge because of the risks involving drug-drug interactions, increased risk of toxicities, and compatibility issues such as mode and frequency of administrations etc. The risk with small molecule therapeutics is particularly high when the small molecule anticancer agents target a ubiquitously expressed protein such as CD-47 as it can lead to serious hematological toxicities. In the context of combination therapy, the risk is even higher when one or more cytotoxic agents are to be used as combination partners.

The present invention, in one aspect, provides a method of treating or delaying the progression of a disease or a disorder mediated by CD47-SIRPα pathway in a human subject, wherein the method comprises the administration, preferably the oral administration, of a CD47-SIRPα blocking agent selected from the group consisting of the compound of formula (I), stereoisomers thereof, and pharmaceutically acceptable salts, solvates, amides and esters of the compound of formula (I) and stereoisomers thereof; as well as the administration of Azacitidine, or a pharmaceutically acceptable salt thereof.

In a second aspect, the present disclosure provides a CD47-SIRPα blocking agent, selected from the group consisting of the compound of formula (I), stereoisomers thereof, and pharmaceutically acceptable salts, solvates, amides and esters of the compound of formula (I) and stereoisomer thereof; for use in a method of treating or delaying progression of a disease or a disorder mediated by CD47-SIRPα pathway in a human subject, wherein the method comprises the administration, preferably the oral administration, of the CD47-SIRPα blocking agent; as well as the administration of Azacitidine, or a pharmaceutically acceptable salt thereof.

In another aspect, the present disclosure provides a kit comprising:
(i) a plurality of pharmaceutical compositions, preferably in unit dosage form, comprising a CD47-SIRPα blocking agent, selected from the group consisting of the compound of formula (I), stereoisomers thereof, and pharmaceutically acceptable salts, solvates, amides and esters of the compound of formula (I) and stereoisomer thereof;
(ii) optionally, a plurality of pharmaceutical compositions, preferably in unit dosage form, comprising Azacitidine, or a pharmaceutically acceptable salt thereof; and,
(iii) a package and/or package insert, said package and/or insert having instructions printed thereon, to use said pharmaceutical compositions in a method of treating or delaying progression of a disease or a disorder mediated by CD47-SIRPα in a subject.

In yet another aspect, the present disclosure provides the use of a CD47-SIRPα blocking agent, selected from the group consisting of the compound of formula (I), stereoisomers thereof, and pharmaceutically acceptable salts, solvates, amides and esters of the compound of formula (I) and stereoisomer thereof; in the manufacture of a pharmaceutical composition or medicament for use in a method of treating or delaying progression of a disease or a disorder mediated by CD47-SIRPα pathway in a human subject, wherein the method comprises the administration, preferably the oral administration, of the CD47-SIRPα blocking agent, as well as the administration of Azacitidine, or a pharmaceutically acceptable salt thereof.

A pharmaceutical composition comprising CD47-SIRPα blocking agent, selected from the group consisting of the compound of formula (I), stereoisomers thereof, and pharmaceutically acceptable salts, solvates, amides and esters of the compound of formula (I) and stereoisomer thereof, for use in combination with another pharmaceutical composition comprising Azacitidine, in a method of treating or delaying progression of a disease or a disorder mediated by CD47-SIRPα pathway in a human subject, wherein the method comprises the administration, preferably the oral administration, of the CD47-SIRPα blocking agent, as well as the administration of Azacitidine, or a pharmaceutically acceptable salt thereof

Particularly preferred embodiments concern methods of treating or delaying the progression of Acute Myeloid Leukemia (AML) or Myelodysplastic Syndrome (MDS), wherein the methods comprise administering to the subject in need thereof of a compound of formula (I) at a total daily dose not exceeding 800 mg, preferably not exceeding 600 mg, more preferably not exceeding 500 mg, most preferably not exceeding 400 mg, or the molar equivalent amount or equipotent amount of a stereoisomer of the compound of formula (I) or of a pharmaceutically acceptable salt, solvate, amide or ester of the compound of formula (I) or stereoisomer thereof. Yet further aspects of the present disclosure provide, amongst others, pharmaceutical compositions, especially in unit dosage form, comprising a CD47-SIRPα blocking agent, selected from the group consisting of the compound of formula (I), stereoisomers thereof, and pharmaceutically acceptable salts, solvates, amides and esters of the compound of formula (I) and stereoisomer thereof, optionally in combination with Azacitidine, or a pharmaceutically acceptable salt thereof. Typically, such pharmaceutical compositions are specifically adapted and/or suited for use in the methods of the present invention.

Specific details and preferred embodiments of the afore mentioned methods as well as of the compositions and pharmaceutical kits used therein will become evident to those skilled in the art on the basis of the following detailed description and experimental information.

### Definitions

The term “stereoisomer” as used herein refers to the field of stereochemistry, stereoisomerism or spatial isomerism. Stereoisomer is a form of isomerism in which molecules have the same molecular formula and sequence of bonded atoms (constitution) but differ in the three-dimensional orientations of their atoms in space. Broadly, the stereoisomer includes enantiomers and diastereomers along with the sub-types thereof, including but not limited to meso compounds, cis-trans isomers, E-Z isomers, non-enantiomeric optical isomers etc.

The term “amide” or “amide derivative(s)” or “carboxamide” as used herein refers to a functional group of general formula R—C(═O)—NR′R″, where R, R′, and R″ represent any group including hydrogen atoms. Amides can be viewed as a derivative of a carboxylic acid R—C(═O)—OH wherein the hydroxyl group (—OH) is replaced by an amine group (—NR′R″).

The term “ester” as used herein refers to a functional group of general formula R—C(═O)—OR′, where R and R′ represents any group and R′ cannot be hydrogen. Esters can be viewed as a derivative of a carboxylic acid R—C(═O)—OH wherein the hydrogen of hydroxyl group (—OH) is replaced by R′.

The term “pharmaceutical composition” as used herein has its conventional meaning and refers to a composition which is pharmaceutically acceptable.

The term “pharmaceutically acceptable” as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio.

The term “salt” as used herein has its conventional meaning and includes the acid addition and base salts of a pharmaceutically active compound.

The term “solvate” as used herein has its conventional meaning and refers to a compound formed by solvation, for example as a combination of solvent molecules with molecules or ions of a solute. Well known solvent molecules include water, alcohols, nitriles and polar organic solvents.

The term “subject” as used herein refers to humans suffering from or at risk for a certain disease or disorder. The term “subject” and “patient” herein are used interchangeably.

The terms “treat”, “treating” or “treatment”, when used in conjunction with a specific disease or symptom (for example: “method of treating disease . . . ”) refer to and/or encompass curing, alleviating or abrogating said disease and/or accompanying symptoms, diminishing extent of disease, stabilizing (i.e. not worsening) the state of disease, delaying or slowing of disease progression, ameliorating the disease state, prolonging survival (as compared to expected survival without treatment), etc. Treatment need not mean that the disease, disorder, or condition is totally cured. To be an effective treatment, a useful composition herein needs only to reduce the severity of a disease, disorder, or condition, reduce the severity of one or more symptoms associated therewith, or provide improvement to a patient or subject's quality of life. The terms “treat”, “treating” or “treatment”, when used in relation to a patient or subject (for example: “method of treating a subject”), typically refers to the act of administering a therapeutic compound to said patient or subject for whatever therapeutic and/or prophylactic purpose.

The terms “prevent”, “preventing” or “prevention”, as used herein, refer to and/or encompass the delay, prevention, suppression, or inhibition of the onset of a disease, disorder, or condition. As used in accordance with the presently described subject matter, the term “prevention” relates to a process of prophylaxis in which a subject is given the presently described compositions or formulations prior to the induction or onset of the disease/disorder process. The term “suppression” is used to describe a condition wherein the disease/disorder process has already begun but obvious symptoms of the condition have yet to manifest. Thus, an individual may have the disease/disorder, but no outside signs of the disease/disorder have yet been clinically recognized. In either case, the term prophylaxis can be applied to encompass both prevention and suppression.

The term “molar equivalent” or “equimolar” in the context of a salt, solvate, amide and ester, or stereoisomers of the compound of formula (I) refers to the moles or molar concentration equivalent to corresponding free acid or base of the compound of formula (I), or stereoisomer thereof.

The term “equipotent” in the context of a salt, solvate, amide and ester, or stereoisomers of the compound of formula (I) refers to a dose or dosage that has an equivalent potency as the corresponding free acid or base of the compound of formula (I), or stereoisomer thereof. It is also common in the art to refer to amounts of a given compound ‘equivalent’ to a specified amount of a reference compound. In expressing dose amounts in the label and/or product information of authorized medicinal products comprising a salt form of an active compound that can also be used in free base form, it is customary practice to specify the dose of the free base to which the dose of the salt as used is equivalent. In this context, the term ‘equipotent’ is deemed synonymous to the term ‘equivalent’.

### DESCRIPTION OF FIGURES

FIGS. 1-3 describe % reduction in BLAST cells after administration of Calcium salt of compound of formula (I) 200 mg, 300 mg and 400 mg BID, respectively in combination with Azacitidine. Efficacy is measured in terms of BLAST % change. X axis denotes randomized patient no., while Y axis denotes BLAST %.

FIG. 4 describes effect of calcium salt of compound of formula (I) on phagocytosis of HL-60 cells by human macrophages.

### DETAILED DESCRIPTION

### Compounds and Pharmaceutical Compositions

As defined herein before, the methods of the present disclosure comprise the administration of the CD47-SIRPα blocking agent, typically in the form of a pharmaceutical composition or in unit dosage form, wherein said CD47-SIRPα blocking agents, selected from the group consisting of the compound of formula (I):

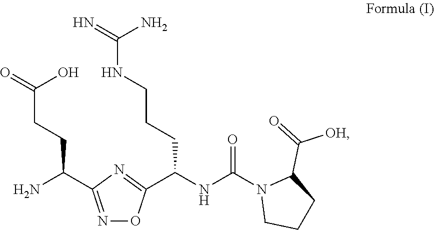

stereoisomers thereof, and pharmaceutically acceptable salts, solvates, amides and esters of the compound of formula (I) and stereoisomer thereof.

As already explained before, the compound of formula (I) has been disclosed in PCT publication WO2019/138367, which also teaches methods for preparing this compound.

In one embodiment, the pharmaceutically acceptable salt of the compound of formula (I) is selected from a Calcium, Magnesium, Potassium and Sodium salts of the compound of formula (I). In another embodiment, the compound of formula (I) is a Calcium salt of the compound of formula (I). In yet another embodiment, the compound of formula (I) is a Sodium salt of the compound of formula (I).

In a particularly preferred embodiment, the CD47-SIRPα blocking agent is selected from the group consisting of the compound of formula (I), pharmaceutically acceptable salts thereof, solvates and hydrates thereof, more preferably from the group consisting of the compound of formula (I), the calcium salt thereof and solvates and hydrates thereof.

The present disclosure provides the CD47-SIRPα blocking agent in the form of a pharmaceutical composition. As used herein, the term “pharmaceutical composition” has its conventional meaning and refers to a composition which is pharmaceutically acceptable. The term ‘pharmaceutically acceptable’ as used herein has its conventional meaning and refers to compounds, materials, compositions and/or dosage forms, which are, within the scope of sound medical judgment can be administered to a human and/or placed in contact with the tissues of humans, without excessive toxicity, irritation, allergic response and other complications, commensurate with a reasonable benefit/risk ratio. The pharmaceutical composition of the present disclosure comprises, besides the CD47-SIRPα blocking agent, one or more additional ingredients. In a preferred embodiment, the composition comprises one or more additional ingredients that render the composition suitable for administration to subjects in need thereof, such as one or more carriers and/or excipients. As is known by those of average skill in the art, the appropriate choice of excipients is largely dependent on the pharmaceutical form and route of administration preferred. The compositions of the present disclosure can be formulated for a variety of routes of administration, oral administration being particularly preferred. It is within the purview of those of average skill in the art to conceive and develop suitable formulations, relying on the common general knowledge as reflected in text books such as Remington's Pharmaceutical Sciences (Meade Publishing Co., Easton, Pa., 20^{th} Ed., 2000), the entire disclosure of which is herein incorporated by reference, and routine development efforts.

In accordance with the various aspects of the present disclosure, the pharmaceutical composition is preferably provided in a unit dosage form. Hence, the present disclosure also provides the CD47-SIRPα blocking agent in unit dosage form. The term ‘unit dosage form’ refers to a physically discrete unit suitable as a unitary dosage for human subjects, each unit containing a predetermined quantity of active material calculated to produce the desired therapeutic effect in association with any suitable pharmaceutical carrier(s) and/or excipient(s). Exemplary, non-limiting unit dosage forms include a tablet (e.g., a chewable tablet), caplet, capsule (e.g., a hard capsule or a soft capsule), lozenge, film, strip, gelcap as well as any metered volume of a solution, suspension, syrup or elixir or the like, which may be contained, for instance in a vial, syringe, applicator device, sachet, spray, micropump etc. In accordance with particularly preferred embodiments of the present disclosure, the unit dosage form, is a unit dosage form that is suitable for oral administration, preferably a tablet or capsule.

In one embodiment, the pharmaceutical composition is provided in a unit dosage form as defined herein, which unit dosage form comprises the CD47-SIRPα blocking agent at an amount not exceeding 800 mg, preferably not exceeding 600 mg, more preferably not exceeding 500 mg, most preferably not exceeding 400 mg. In one embodiment, the pharmaceutical composition is provided in a unit dosage form as defined herein, which unit dosage form comprises the CD47-SIRPα blocking agent at an amount of 50 mg to 800 mg. In another embodiment, the pharmaceutical composition is provided in a unit dosage form as defined herein, which unit dosage form comprises the CD47-SIRPα blocking agent at an amount of 50 mg to 100 mg. In another embodiment, the amount of the CD47-SIRPα blocking agent is 50 mg to 200 mg. In another embodiment, the amount of the CD47-SIRPα blocking agent is 50 mg to 400 mg. In another embodiment, the amount of the CD47-SIRPα blocking agent is 50 mg to 800 mg. In another embodiment, the amount of the CD47-SIRPα blocking agent is 100 mg to 200 mg. In another embodiment, the amount of the CD47-SIRPα blocking agent is 100 mg to 400 mg. In another embodiment, the amount of the CD47-SIRPα blocking agent is 100 mg to 800 mg. In another embodiment, the amount of the CD47-SIRPα blocking agent is 200 mg to 400 mg. In another embodiment, the amount of the CD47-SIRPα blocking agent is 200 mg to 800 mg. In another embodiment, the amount of the CD47-SIRPα blocking agent is 400 mg to 800 mg. In another embodiment, the pharmaceutical composition is provided in a unit dosage form as defined herein, which unit dosage form comprises the CD47-SIRPα blocking agent at an amount of 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg or 800 mg. In another embodiment, the amount of the CD47-SIRPα blocking agent is 50 mg. In another embodiment, the amount of the CD47-SIRPα blocking agent is 100 mg. In another embodiment, the amount of the CD47-SIRPα blocking agent is 200 mg. In another embodiment, the amount of the CD47-SIRPα blocking agent is 400 mg. In another embodiment, the amount of the CD47-SIRPα blocking agent is 800 mg.

In one embodiment, the pharmaceutical composition is provided in a unit dosage form as defined herein, which unit dosage form comprises the compound of formula (I) at an amount not exceeding 800 mg, preferably not exceeding 600 mg, more preferably not exceeding 500 mg, most preferably not exceeding 400 mg, or a stereoisomer of the compound of formula (I) or a salt, solvate, amide or ester of the compound of formula (I) or stereoisomer thereof, at the molar equivalent amount. In one embodiment, the pharmaceutical composition is provided in a unit dosage form as defined herein, which unit dosage form comprises the compound of formula (I) at an amount of 50 mg to 800 mg, or a stereoisomer of the compound of formula (I) or a salt, solvate, amide or ester of the compound of formula (I) or stereoisomer thereof, at the molar equivalent amount. In another embodiment, the pharmaceutical composition is provided in a unit dosage form as defined herein, which unit dosage form comprises the compound of formula (I) at an amount of 50 mg to 100 mg; 50 mg to 200 mg; 50 mg to 400 mg; 50 mg to 800 mg; 100 mg to 200 mg; 100 mg to 400 mg; 100 mg to 800 mg; 200 mg to 400 mg; 200 mg to 800 mg; or 400 mg to 800 mg, or a stereoisomer of the compound of formula (I) or a salt, solvate, amide or ester of the compound of formula (I) or stereoisomer thereof, at the molar equivalent amount. In another embodiment, the pharmaceutical composition is provided in a unit dosage form as defined herein, which unit dosage form comprises the compound of formula (I) at an amount of 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg or 800 mg, or a stereoisomer of the compound of formula (I) or a salt, solvate, amide or ester of the compound of formula (I) or stereoisomer thereof, at the molar equivalent amount. In other embodiments, the pharmaceutical composition is provided in a unit dosage form as defined herein, which unit dosage form comprises the compound of formula (I) is 50 mg, 100 mg, 200 mg, 400 mg or 800 mg, or a stereoisomer of the compound of formula (I) or a salt, solvate, amide or ester of the compound of formula (I) or stereoisomer thereof, at the molar equivalent amount.

In one embodiment, the pharmaceutical composition is provided in a unit dosage form as defined herein, which unit dosage form comprises the compound of formula (I) at an amount not exceeding 800 mg, preferably not exceeding 600 mg, more preferably not exceeding 500 mg, most preferably not exceeding 400 mg, or a stereoisomer of the compound of formula (I) or a salt, solvate, amide or ester of the compound of formula (I) or stereoisomer thereof, at the equipotent amount. In one embodiment, the pharmaceutical composition is provided in a unit dosage form as defined herein, which unit dosage form comprises the compound of formula (I) at an amount of 50 mg to 800 mg, or a stereoisomer of the compound of formula (I) or a salt, solvate, amide or ester of the compound of formula (I) or stereoisomer thereof, at the equipotent amount. In another embodiment, the pharmaceutical composition is provided in a unit dosage form as defined herein, which unit dosage form comprises the compound of formula (I) at an amount of 50 mg to 100 mg; 50 mg to 200 mg; 50 mg to 400 mg; 50 mg to 800 mg; 100 mg to 200 mg; 100 mg to 400 mg; 100 mg to 800 mg; 200 mg to 400 mg; 200 mg to 800 mg; or 400 mg to 800 mg, or a stereoisomer of the compound of formula (I) or a salt, solvate, amide or ester of the compound of formula (I) or stereoisomer thereof, at the equipotent amount. In another embodiment, the pharmaceutical composition is provided in a unit dosage form as defined herein, which unit dosage form comprises the compound of formula (I) at an amount of 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg or 800 mg, or a stereoisomer of the compound of formula (I) or a salt, solvate, amide or ester of the compound of formula (I) or stereoisomer thereof, at the equipotent amount. In other embodiments, the pharmaceutical composition is provided in a unit dosage form as defined herein, which unit dosage form comprises the compound of formula (I) is 50 mg, 100 mg, 200 mg, 400 mg or 800 mg, or a stereoisomer of the compound of formula (I) or a salt, solvate, amide or ester of the compound of formula (I) or stereoisomer thereof, at the equipotent amount.

In one embodiment, the pharmaceutical composition is provided in a unit dosage form as defined herein, which unit dosage form comprises the compound of formula (I) at an amount not exceeding 800 mg, preferably not exceeding 600 mg, more preferably not exceeding 500 mg, most preferably not exceeding 400 mg. In one embodiment, the pharmaceutical composition is provided in a unit dosage form as defined herein, which unit dosage form comprises the compound of formula (I) at an amount of 50 mg to 800 mg. In another embodiment, the pharmaceutical composition is provided in a unit dosage form as defined herein, which unit dosage form comprises the compound of formula (I) at an amount of 50 mg to 100 mg; 50 mg to 200 mg; 50 mg to 400 mg; 50 mg to 800 mg; 100 mg to 200 mg; 100 mg to 400 mg; 100 mg to 800 mg; 200 mg to 400 mg; 200 mg to 800 mg; or 400 mg to 800 mg. In another embodiment, the pharmaceutical composition is provided in a unit dosage form as defined herein, which unit dosage form comprises the compound of formula (I) at an amount of 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg or 800 mg. In other embodiments, the pharmaceutical composition is provided in a unit dosage form as defined herein, which unit dosage form comprises the compound of formula (I) is 50 mg, 100 mg, 200 mg, 400 mg or 800 mg.

In one embodiment, the pharmaceutical composition is provided in a unit dosage form as defined herein, which unit dosage form comprises the calcium salt of the compound of formula (I) at an amount not exceeding 800 mg, preferably not exceeding 600 mg, more preferably not exceeding 500 mg, most preferably not exceeding 400 mg. In one embodiment, the pharmaceutical composition is provided in a unit dosage form as defined herein, which unit dosage form comprises the calcium salt of the compound of formula (I) at an amount of 50 mg to 800 mg. In another embodiment, the pharmaceutical composition is provided in a unit dosage form as defined herein, which unit dosage form comprises the calcium salt of the compound of formula (I) at an amount of 50 mg to 100 mg; 50 mg to 200 mg; 50 mg to 400 mg; 50 mg to 800 mg; 100 mg to 200 mg; 100 mg to 400 mg; 100 mg to 800 mg; 200 mg to 400 mg; 200 mg to 800 mg; or 400 mg to 800 mg. In another embodiment, the pharmaceutical composition is provided in a unit dosage form as defined herein, which unit dosage form comprises the calcium salt of the compound of formula (I) at an amount of 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg or 800 mg. In other embodiments, the pharmaceutical composition is provided in a unit dosage form as defined herein, which unit dosage form comprises the calcium salt of the compound of formula (I) is 50 mg, 100 mg, 200 mg, 400 mg or 800 mg.

In accordance with certain embodiments of the present disclosure, the pharmaceutical composition as defined herein, further comprises azacitidine or a pharmaceutically acceptable salt thereof as a further active pharmaceutical ingredient. Azacitidine is the international non-proprietary name (INN) of the compound having the IUPAC name 4-Amino-1-β-D-ribofuranosyl-s-triazin-2(1H)-one and the following structural formula (II).

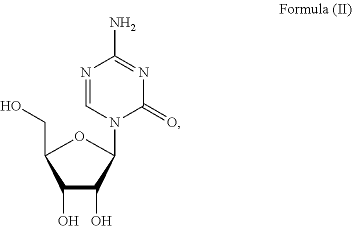

In accordance with the present disclosure, Azacitidine may be used in free base form as well as in the form of a pharmaceutically acceptable salt. Salts suitable for the purposes of the present disclosure include acid addition salts, such as salts formed with an acid having a pKₐ of about 5 or less, preferably an acid selected from the group consisting of hydrochloric, L-lactic, acetic, phosphoric, (+)-L-tartaric, citric, propionic, butyric, hexanoic, L-aspartic, L-glutamic, succinic, EDTA, maleic, methanesulfonic acid, HBr, HF, HI, nitric, nitrous, sulfuric, sulfurous, phosphorous, perchloric, chloric, chlorous acid, carboxylic acid, sulfonic acid, ascorbic, carbonic, and fumaric acid.

Medicinal products comprising Azacitidine as the sole active pharmaceutical ingredient have received regulatory approval for the treatment of (adult) patients with acute myeloid leukaemia (AML), higher-risk myelodysplastic syndromes (MDS) and chronic myelomonocytic leukaemia (CMML), via oral administration as well as via subcutaneous injection. It will be understood by those skilled in the art, based on the present teachings, that it may be beneficial, in certain embodiments, to provide the CD47-SIRPα blocking agent and azacitidine, or a pharmaceutically acceptable salt of Azacitidine, in the form of an oral fixed dose combination (FDC) product, in particular in the form of a unit dosage form, such as a tablet or capsule, as described herein, comprising azacitidine or a pharmaceutically acceptable salt thereof, in addition to the CD47-SIRPα blocking agent.

In accordance with preferred embodiments of the present disclosure, the pharmaceutical composition is provided in a unit dosage form as defined herein, which unit dosage form comprises the Azacitidine or a pharmaceutically acceptable salt thereof in an amount of at least about 25 mg, more preferably at least about 50 mg, at least about 75 mg, at least about 100 mg, at least about 125, at least about 150 mg, at least about 175 mg or at least about 200 mg. In accordance with the various aspects of the present disclosure, the composition is provided in a unit dosage form comprising Azacitidine or a pharmaceutically acceptable salt thereof in an amount of about 500 mg or less, more preferably about 450 mg or less, about 400 mg or less, about 350 mg or less, about 325 mg or less or about 300 mg or less. In accordance with embodiments of the present disclosure, the pharmaceutical composition is provided in a unit dosage form comprising Azacitidine or a pharmaceutically acceptable salt thereof in an amount of about 100-400 mg, more preferably in an amount of about 150-350 mg, most preferably in an amount of about 200-300 mg, e.g. in an amount of about 200 mg or in an amount of about 300 mg of Azacitidine or a pharmaceutically acceptable salt thereof.

In accordance with preferred embodiments of the present disclosure, the pharmaceutical composition is provided in a unit dosage form as defined herein, which unit dosage form comprises the Azacitidine in an amount of at least about 25 mg, more preferably at least about 50 mg, at least about 75 mg, at least about 100 mg, at least about 125, at least about 150 mg, at least about 175 mg or at least about 200 mg, or a pharmaceutically acceptable salt of Azacitidine in the equimolar or equipotent amount. In accordance with the various aspects of the present disclosure, the composition is provided in a unit dosage form comprising Azacitidine in an amount of about 500 mg or less, more preferably about 450 mg or less, about 400 mg or less, about 350 mg or less, about 325 mg or less or about 300 mg or less, or a pharmaceutically acceptable salt of Azacitidine in the equimolar or equipotent amount. In accordance with embodiments of the invention, the pharmaceutical composition is provided in a unit dosage form comprising Azacitidine in an amount of about 100-400 mg, more preferably in an amount of about 150-350 mg, most preferably in an amount of about 200-300 mg, e.g. in an amount of about 200 mg or in an amount of about 300 mg or a pharmaceutically acceptable salt of Azacitidine in the equimolar or equipotent amount.

### Therapeutic Indications

The methods of the present disclosure, comprising the administration of the CD47-SIRPα blocking agent, typically in the form of a pharmaceutical composition or unit dosage form as defined herein before, are intended to, directed at and/or effective in treating or delaying the progression of a disease or a disorder mediated by CD47-SIRPα pathway.

In one embodiment, the disease or disorder mediated by CD47-SIRPα pathway is a cancer.

In one embodiment, the disease or disorder mediated by CD47-SIRPα pathway is a hematological cancer.

In one embodiment, the disease or disorder mediated by CD47-SIRPα pathway is leukemia.

In one embodiment, the disease or disorder mediated by CD47-SIRPα pathway is Acute Myeloid Leukemia (AML) or Myelodysplastic syndrome (MDS).

In yet another embodiment, AML is a newly diagnosed AML or a relapsed and/or refractory AML.

In a preferred embodiment of the present disclosure, the disease or disorder mediated by CD47-SIRPα pathway is Acute Myeloid Leukemia (AML), wherein the treatment is characterized by the treated subject achieving at least one of the following response criteria according to European Leukemia Net (ELN-2017) after at least one treatment cycle comprising 28 days of treatment:
a. Complete remission;
b. Complete remission with incomplete hematologic recovery;
c. Morphologic leukemia free state;
d. Partial remission; or
e. Stable Disease lasting 3 months or more

In one embodiment, the disease or disorder mediated by CD47-SIRPα pathway is Myelodysplastic syndrome (MDS), and wherein the treatment is characterized by the treated subject achieving at least one of the following response criteria according to International Working Group (IWG) 2006:
a. Complete remission;
b. Partial remission;
c. Stable disease; or
d. Hematological Improvement

### Subjects to be Treated

The methods of the present disclosure treat or delay the progression of a disease, or a disorder mediated by CD47-SIRPα pathway in a human subject. Hence, in preferred embodiments of the present disclosure, the human subject is a subject suffering from a disease or disorder mediated by CD47-SIRPα pathway. In one embodiment, the human subject is a subject suffering from a relapsing or refractory form of a disease or disorder mediated by CD47-SIRPα pathway. In one embodiment, the human subject is a subject at risk of suffering from a disease or disorder mediated by CD47-SIRPα pathway, especially a subject at risk of suffering from a relapse of a disease or disorder mediated by CD47-SIRPα pathway. Diseases or disorders mediated by CD47-SIRPα pathway can in principle occur and/or manifest at any stage in life but are most common in older adults. Hence, the subject to be treated in accordance with the present disclosure can typically have any age above 12 years, such an age ranging from 12-99 years. However, in preferred embodiments of the present disclosure the subject has an age of 40 years or older, such as 50 years or older, 55 years or older, 60 years or older or 65 years or older. In one embodiment of the present disclosure, the subject is human male. In another embodiment of the present disclosure, the subject is human female.

In one embodiment, the subject is a subject suffering from Acute myeloid leukemia (AML). In one embodiment, the subject is a subject diagnosed with AML. In one embodiment of the present disclosure, the subject has a positive diagnosis of AML according to the World Health Organization (WHO 2016) criteria. The WHO 2016 criteria divide AML into several groups. These include AML with certain genetic abnormalities, AML with myelodysplasia-related changes, AML related to previous chemotherapy or radiation, AML with minimal differentiation, AML without maturation, AML with maturation, acute myelomonocytic leukemia, acute monoblastic/monocytic leukemia, pure erythroid leukemia, acute megakaryoblastic leukemia, acute basophilic leukemia, acute panmyelosis with fibrosis, myeloid sarcoma (also known as granulocytic sarcoma or chloroma), myeloid proliferations related to down syndrome [Arber D A, Orazi A, Hasserjian R, et al. The 2016 revision to the World Health Organization classification of myeloid neoplasms and acute leukemia. Blood. 2016; 127(20):2391-2405].

In one embodiment, the subject is a subject newly diagnosed with AML. In one embodiment, the human subject is a subject suffering from a relapsed and/or refractory AML. In one embodiment, the human subject is a subject suffering from a relapsed and/or refractory AML has received at least one line of previous therapy. In one embodiment, the human subject is a subject suffering from a relapsed and/or refractory AML has received at least one line of previous therapy and is eligible for one or more second or later line(s) of treatment. In one embodiment, wherein at least one line of previous therapy does not comprise a hypomethylating agent. In one embodiment, one of the second or later line of treatments is a therapeutically effective dose of Azacitidine, or a pharmaceutically acceptable salt thereof. In one embodiment, the subjects is a subject diagnosed with relapsed/refractory AML who has received at least one line of previous therapy other than hypomethylating agents, and is eligible for receiving Azacitidine. In one embodiment, the subject is a newly diagnosed subject suffering from AML that is not eligible for intensive chemotherapy.

In one embodiment, the subject is a subject suffering from Myelodysplastic syndrome (MDS). In one embodiment, the subject is a subject diagnosed with MDS. In one embodiment of the present disclosure, the subject has a positive diagnosis of MDS according to the World Health Organization (WHO 2016) criteria. The WHO 2016 criteria divide MDS into several groups. These include, MDS with single lineage dysplasia, MDS with multilineage dysplasia MDS with ring sideroblasts (MDS-RS), MDS with excess blasts, MDS with isolated del(5q), MDS, unclassifiable (MDS-U) [Arber D A, Orazi A, Hasserjian R, et al. The 2016 revision to the World Health Organization classification of myeloid neoplasms and acute leukemia. Blood. 2016; 127(20):2391-2405].

The IWG criteria 2006 define 4 aspects of responses based on treatment goals: (1) altering the natural history of the disease, (2) cytogenetic response, (3) hematologic improvement (HI), and (4) QOL as provided in the below:

International Working Group (IWG) response criteria 2006 for altering natural history of MDS

| Category | Response criteria (responses must last at least 4 week) |
|---|---|
| Complete | Bone marrow: ≤5% myeloblasts with normal maturation of all cell lines* |
| remission | Persistent dysplasia will be noted*† |
| | Peripheral blood‡ |
| | Hgb ≥ 11 g/dL |
| | Platelets ≥ 100 × 10⁹/L |
| | Neutrophils ≥ 1.0 × 10⁹/L† |
| | Blasts 0% |
| Partial | All CR criteria if abnormal before treatment except: |
| remission | Bone marrow blasts decreased by ≥50% over pretreatment but still >5% |
| | Cellularity and morphology not relevant |
| Marrow | Bone marrow: ≤5% myeloblasts and decrease by ≥50% over pretreatment† |
| CR† | Peripheral blood: if HI responses, they will be noted in addition to marrow CR† |
| Stable | Failure to achieve at least PR, but no evidence of progression for >8 weeks |
| disease | |
| Failure | Death during treatment or disease progression characterized by worsening of |
| | cytopenias, increase in percentage of bone marrow blasts, or progression to a |
| | more advanced MDS FAB subtype than pretreatment |
| Relapse | At least 1 of the following: |
| after CR or | Return to pretreatment bone marrow blast percentage |
| PR | Decrement of ≥50% from maximum remission/response levels in |
| | granulocytes or platelets |
| | Reduction in Hgb concentration by ≥1.5 g/dL or transfusion dependence |
| Cytogenetic | Complete |
| response | Disappearance of the chromosomal abnormality without appearance of new |
| | ones |
| | Partial |
| | At least 50% reduction of the chromosomal abnormality |
| Disease | For patients with: |
| progression | Less than 5% blasts: ≥50% increase in blasts to >5% blasts |
| | 5%-10% blasts: ≥50% increase to >10% blasts |
| | 10%-20% blasts: ≥50% increase to >20% blasts |
| | 20%-30% blasts: ≥50% increase to >30% blasts |
| | Any of the following: |
| | At least 50% decrement from maximum remission/response in granulocytes or |
| | platelets |
| | Reduction in Hgb by ≥2 g/dL |
| | Transfusion dependence |
| Survival | Endpoints: |
| | Overall: death from any cause |
| | Event free: failure or death from any cause |
| | PFS: disease progression or death from MDS |
| | DFS: time to relapse |
| | Cause-specific death: death related to MDS |

To convert hemoglobin from grams per deciliter to grams per liter, multiply grams per deciliter by 10.
MDS indicates myelodysplastic syndromes; Hgb, hemoglobin; CR, complete remission; HI, hematologic improvement; PR, partial remission; FAB, French-American-British; AML, acute myeloid leukemia; PFS, progression-free survival; DFS, disease-free survival.
* Dysplastic changes should consider the normal range of dysplastic changes (modification).
† Modification to IWG response criteria.
‡ In some circumstances, protocol therapy may require the initiation of further treatment (eg, consolidation, maintenance) before the 4-week period. Such patients can be included in the response category into which they fit at the time the therapy is started. Transient cytopenias during repeated chemotherapy courses should not be considered as interrupting durability of response, as long as they recover to the improved counts of the previous course. [Bruce D. Cheson et al. Clinical application and proposal for modification of the International Working Group (IWG) response criteria in myelodysplasia Blood (2006) 108 (2): 419-425].

In one embodiment, the subject is a subject newly diagnosed with MDS. In one embodiment, the human subject is a subject suffering from a relapsed and/or refractory MDS.

In one embodiment, the human subject is a subject suffering from a relapsed and/or refractory MDS has received at least one line of previous therapy. In one embodiment, the subject suffering from MDS is an intermediate risk, high risk, or a very high-risk MDS subject. In one embodiment, the subject suffering from MDS is an intermediate risk, high risk, or a very high-risk MDS subject who is eligible to receive Azacitidine. In an embodiment, the subject suffering from MDS has a Revised International Prognostic Scoring System (IPSS-R)≥3.5. In an embodiment, the subject has not been previously treated with a hypomethylating agent. In an embodiment, the subject suffering from MDS is eligible for one or more second or later line(s) of treatment. In an embodiment, the one of the second or later line of treatments is a therapeutically effective dose of Azacitidine, or a pharmaceutically acceptable salt thereof. IPSS-R is a prognostic score values, and is determined by combining the scores of five disease factors: BM Blasts, Cytogenetics, Hemoglobin, Platelet count and Absolute neutrophil count (ANC) [Peter L. Greenberg et al. Revised International Prognostic Scoring System for Myelodysplastic Syndromes. Blood. 2012 Sep. 20; 120(12): 2454-2465].

In one embodiment, the human subject has an Eastern Cooperative Oncology Group (ECOG) Performance Status Scale of 0-2. ECOG performance status scale assesses a patient's disease progression, daily living abilities, and guides treatment decisions based on a 5-point scale ranging from grade 0 to grade 5 as defined below:

| ECOG grade | Performance status |
|---|---|
| 0 | Fully active, able to carry on all pre-disease performance without |
| | restriction |
| 1 | Restricted in physically strenuous activity but ambulatory and able to carry |
| | out work of a light or sedentary nature, e.g., light house work, office work |
| 2 | Ambulatory and capable of all selfcare but unable to carry out any work |
| | activities; up and about more than 50% of waking hours |
| 3 | Capable of only limited selfcare; confined to bed or chair more than 50% |
| | of waking hours |
| 4 | Completely disabled; cannot carry on any selfcare; totally confined to bed |
| | or chair |
| 5 | Dead |

In another embodiment, the human subject meets at least one of the criteria a) and b), preferably the human subject meets both criteria a) and b):
a) bone marrow assessment characterized by:
   i. WBC<20,000/μL, wherein hydroxyurea can be given to bring WBC count to <20,000/μl;
   ii. Platelet count≥50,000/μL without transfusion support; and/or
   iii. Hemoglobin≥9 g/dL, wherein transfusion is allowed to achieve this Hb; and/or,
b) organ function characterized by:
i. Total Bilirubin≤1.5×Upper Limit Normal (ULN); or ≤2.5×ULN if the subject has Gilbert's syndrome;
ii. AST (SGOT)≤3×ULN (≤5×ULN if the subject has liver metastases);
iii. ALT (SGPT)≤3×ULN (≤5×ULN if the subject liver metastases);
iv. Creatinine clearance (CrCl)≥60 mL/min; and/or
v. Albumin≥3.0 g/dL.

### Treatment Regimens

The methods of the present disclosure, treating or delaying the progression of a disease or a disorder mediated by CD47-SIRPα pathway in a human subject, comprise the administration of the CD47-SIRPα blocking agent, typically in the form of any of the pharmaceutical compositions or unit dosage forms defined herein elsewhere.

In one embodiment, the method comprises the oral administration of the CD47-SIRPα blocking agent at a total daily dose not exceeding 800 mg, preferably not exceeding 600 mg, more preferably not exceeding 500 mg, most preferably not exceeding 400 mg.

In one embodiment, the method comprises the oral administration of the CD47-SIRPα blocking agent at a total daily dose of 50 mg to 800 mg. In another embodiment, the method comprises the oral administration of the CD47-SIRPα blocking agent at an amount of 50 mg to 100 mg. In another embodiment, the total daily dose of the CD47-SIRPα blocking agent is 50 mg to 200 mg. In another embodiment, the total daily dose of the CD47-SIRPα blocking agent is 50 mg to 400 mg. In another embodiment, the total daily dose of the CD47-SIRPα blocking agent is 50 mg to 800 mg. In another embodiment, the total daily dose of the CD47-SIRPα blocking agent is 100 mg to 200 mg. In another embodiment, the total daily dose of the CD47-SIRPα blocking agent is 100 mg to 400 mg. In another embodiment, the total daily dose of the CD47-SIRPα blocking agent is 100 mg to 800 mg. In another embodiment, the total daily dose of the CD47-SIRPα blocking agent is 200 mg to 400 mg. In another embodiment, the total daily dose of the CD47-SIRPα blocking agent is 200 mg to 800 mg. In another embodiment, the total daily dose of the CD47-SIRPα blocking agent is 400 mg to 800 mg. In another embodiment, the method comprises the oral administration of the CD47-SIRPα blocking agent at a total daily dose of 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg or 800 mg. In another embodiment, the total daily dose of the CD47-SIRPα blocking agent is 50 mg. In another embodiment, the total daily dose of the CD47-SIRPα blocking agent is 100 mg. In another embodiment, the total daily dose of the CD47-SIRPα blocking agent is 200 mg. In another embodiment, the total daily dose of the CD47-SIRPα blocking agent is 400 mg. In another embodiment, the total daily dose of the CD47-SIRPα blocking agent is 800 mg.

In one embodiment, the method comprises the oral administration of the compound of formula (I) at a total daily dose not exceeding 800 mg, preferably not exceeding 600 mg, more preferably not exceeding 500 mg, most preferably not exceeding 400 mg, or the oral administration of a stereoisomer of the compound of formula (I) or a salt, solvate, amide or ester of the compound of formula (I) or stereoisomer thereof, at the molar equivalent dose. In one embodiment, the method comprises the oral administration of a compound of formula (I) at a total daily dose of 50 mg to 800 mg, or the oral administration of a stereoisomer of the compound of formula (I) or a salt, solvate, amide or ester of the compound of formula (I) or stereoisomer thereof, at the molar equivalent dose. In another embodiment, the method comprises the oral administration of a compound of formula (I) at a total daily dose of 50 mg to 100 mg; 50 mg to 200 mg; 50 mg to 400 mg; 50 mg to 800 mg; 100 mg to 200 mg; 100 mg to 400 mg; 100 mg to 800 mg; 200 mg to 400 mg; 200 mg to 800 mg; or 400 mg to 800 mg, or the oral administration of a stereoisomer of the compound of formula (I) or a salt, solvate, amide or ester of the compound of formula (I) or stereoisomer thereof, at the molar equivalent dose. In another embodiment, the method comprises the oral administration of a compound of formula (I) at an total daily dose of 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg or 800 mg, or the oral administration of a stereoisomer of the compound of formula (I) or a salt, solvate, amide or ester of the compound of formula (I) or stereoisomer thereof, at the molar equivalent dose. In other embodiments, the method comprises the oral administration of the compound of formula (I) at a total daily dose of 50 mg, 100 mg, 200 mg, 400 mg or 800 mg, or the oral administration of a stereoisomer of the compound of formula (I) or a salt, solvate, amide or ester of the compound of formula (I) or stereoisomer thereof, at the molar equivalent dose.

In one embodiment, the method comprises the oral administration of the compound of formula (I) at a total daily dose not exceeding 800 mg, preferably not exceeding 600 mg, more preferably not exceeding 500 mg, most preferably not exceeding 400 mg, or the oral administration of a stereoisomer of the compound of formula (I) or a salt, solvate, amide or ester of the compound of formula (I) or stereoisomer thereof, at the equipotent dose. In one embodiment, the method comprises the oral administration of the compound of formula (I) at a total daily dose of 50 mg to 800 mg, or the oral administration of a stereoisomer of the compound of formula (I) or a salt, solvate, amide or ester of the compound of formula (I) or stereoisomer thereof, at the equipotent dose. In another embodiment, the method comprises the oral administration of the compound of formula (I) at a total daily dose of 50 mg to 100 mg; 50 mg to 200 mg; 50 mg to 400 mg; 50 mg to 800 mg; 100 mg to 200 mg; 100 mg to 400 mg; 100 mg to 800 mg; 200 mg to 400 mg; 200 mg to 800 mg; or 400 mg to 800 mg, or the oral administration of a stereoisomer of the compound of formula (I) or a salt, solvate, amide or ester of the compound of formula (I) or stereoisomer thereof, at the equipotent dose. In another embodiment, the method comprises the oral administration of the compound of formula (I) at a total daily dose of 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg or 800 mg, or the oral administration of a stereoisomer of the compound of formula (I) or a salt, solvate, amide or ester of the compound of formula (I) or stereoisomer thereof, at the equipotent dose. In other embodiments, the method comprises the oral administration of the compound of formula (I) at a total daily dose of 50 mg, 100 mg, 200 mg, 400 mg or 800 mg, or the oral administration of a stereoisomer of the compound of formula (I) or a salt, solvate, amide or ester of the compound of formula (I) or stereoisomer thereof, at the equipotent dose.

In one embodiment, the method comprises the oral administration of the compound of formula (I) at a total daily dose not exceeding 800 mg, preferably not exceeding 600 mg, more preferably not exceeding 500 mg, most preferably not exceeding 400 mg. In one embodiment, the method comprises the oral administration of the compound of formula (I) at a total daily dose of 50 mg to 800 mg. In another embodiment, the method comprises the oral administration of the compound of formula (I) at a total daily dose of 50 mg to 100 mg; 50 mg to 200 mg; 50 mg to 400 mg; 50 mg to 800 mg; 100 mg to 200 mg; 100 mg to 400 mg; 100 mg to 800 mg; 200 mg to 400 mg; 200 mg to 800 mg; or 400 mg to 800 mg. In another embodiment, the method comprises the oral administration of the compound of formula (I) at a total daily dose of 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg or 800 mg. In other embodiments, the method comprises the oral administration of the compound of formula (I) at a total daily dose of 50 mg, 100 mg, 200 mg, 400 mg or 800 mg.

In one embodiment, the method comprises the oral administration of the calcium salt of the compound of formula (I) at a total daily dose not exceeding 800 mg, preferably not exceeding 600 mg, more preferably not exceeding 500 mg, most preferably not exceeding 400 mg. In one embodiment, the method comprises the oral administration of the calcium salt of the compound of formula (I) at a total daily dose of 50 mg to 800 mg. In another embodiment, the method comprises the oral administration of the calcium salt of the compound of formula (I) at a total daily dose of 50 mg to 100 mg; 50 mg to 200 mg; 50 mg to 400 mg; 50 mg to 800 mg; 100 mg to 200 mg; 100 mg to 400 mg; 100 mg to 800 mg; 200 mg to 400 mg; 200 mg to 800 mg; or 400 mg to 800 mg. In another embodiment, the method comprises the oral administration of the calcium salt of the compound of formula (I) at a total daily dose of 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg or 800 mg. In other embodiments, the method comprises the oral administration of the calcium salt of the compound of formula (I) at a total daily dose of 50 mg, 100 mg, 200 mg, 400 mg or 800 mg.

In accordance with preferred embodiments of the present disclosure, the CD47-SIRPα blocking agent is administered to the subject, typically at the total daily doses and/or in the form of the unit dosage for as defined herein before, once or twice daily. Typically, in case the method comprises the administration of the CD47-SIRPα blocking agent twice daily, then the total daily dose as defined herein above is administered in two equal portions, i.e. in such embodiments, half of the total daily dose is administered each time.

In accordance with certain embodiments of the present disclosure, the method comprises the twice daily administration of the CD47-SIRPα blocking agent at a dose of 100 to 400 mg, preferably 100 mg, 200 mg, 300 mg, or 400 mg. In accordance with preferred embodiments of the present disclosure, the method comprises the twice daily administration of the compound of formula (I) at a dose of 100 to 400 mg, preferably 100 mg, 200 mg, 300 mg, or 400 mg; or the twice daily administration of a stereoisomer of the compound of formula (I) or a salt, solvate, amide or ester of the compound of formula (I) or stereoisomer thereof at the molar equivalent dose or at the equipotent dose.

In accordance with other embodiments of the present disclosure, the method comprises the once daily administration of the CD47-SIRPα blocking agent at a dose of 200 to 800 mg, preferably 200 mg, 400 mg, 600 mg, or 800 mg. In accordance with preferred embodiments of the present disclosure, the method comprises the once daily administration of the compound of formula (I) at a dose of 200 to 800 mg, preferably 200 mg, 400 mg, 600 mg, or 800 mg; or the once daily administration of a stereoisomer of the compound of formula (I) or a salt, solvate, amide or ester of the compound of formula (I) or stereoisomer thereof at the molar equivalent dose or at the equipotent dose.

In accordance with preferred embodiments of the present disclosure, methods comprise the administration of the CD47-SIRPα blocking agent to the subject, typically at the total daily doses and frequencies as defined herein before, for a period of at least 1 week, preferably at least 2 weeks, at least 3 weeks, at least 4 weeks, at least 5 weeks, at least 6 weeks, at least 7 weeks, at least 8 weeks, at least 9 weeks of at least 10 weeks, such as for a period of 1 week to 6 months, a period of 2 weeks to 4 months or a period of 3 to 8 weeks. In accordance with certain embodiments of the present disclosure, the method comprises the twice daily administration of the CD47-SIRPα blocking agent at a dose of 200 mg to 400 mg, preferably 200 mg, 300 mg or 400 mg, for at least 15 days, preferably for at least 28 days. In accordance with certain preferred embodiments of the present disclosure, the method comprises the twice daily administration of the compound of formula (I) at a dose of 200 mg to 400 mg, preferably 200 mg, 300 mg or 400 mg, for at least 15 days, preferably for at least 28 days; or the twice daily administration, for at least 15 days, preferably for at least 28 days, of a stereoisomer of the compound of formula (I) or a salt, solvate, amide or ester of the compound of formula (I) or stereoisomer thereof at the molar equivalent dose or at the equipotent dose.

In some embodiments of the present disclosure, the method comprises a multitude of treatment cycles, e.g. two or more treatment cycles, such as 2, 3, 4, 5 or 6 treatment cycles, wherein each cycle comprises the administration to the subject, typically at the regimens as defined herein before, for a period of at least 1 week, preferably at least 2 weeks, at least 3 weeks, at least 4 weeks, at least 5 weeks, at least 6 weeks, at least 7 weeks, at least 8 weeks, at least 9 weeks of at least 10 weeks, such as for a period of 1 week to 6 months, a period of 2 weeks to 4 months or a period of 3 to 8 weeks. The period of time elapsing between consecutive cycles may range from e.g. a period of 1 week to 6 months, a period of 2 weeks to 4 months or a period of 3 to 8 weeks.

In some embodiments of the present disclosure, the method comprises administration of the CD47-SIRPα blocking agent to the human subject before food intake, on an empty stomach and/or at least two hours after food intake. Preferably, the method comprises administration of the CD47-SIRPα blocking agent to the human subject within one hour after food intake.

In certain embodiments, the method further comprises the step of modifying the treatment in case the subject shows signs of a prohibitive toxicity and/or disease progression.

In one embodiment, the prohibitive toxicity is a dose limiting toxicity (DLT). In one embodiment, the DLT is characterized by one or more of the toxicities selected from:
a. Positive for Hy's law (AST or ALT≥3×ULN with concomitant Total Bilirubin≥2×ULN, without findings of cholestasis on radiological scans and no other reasons for increase in AST/ALT and bilirubin);
b. Grade 4 neutropenia (ANC<500/μL) in the absence of persistent disease (AML/MDS); or
c. Any other Grade 3 or higher clinically significant toxicity (except alopecia or nail changes), as graded by National Cancer Institute Common Terminology Criteria for Adverse Events (NCI CTCAE) v 5.0 which is considered as “not clearly or incontrovertibly related to the underlying malignancy, any other co-morbidities or concomitant medications or extraneous causes”.

In one embodiment, the disease progression for a subject suffering from AML is characterized by the treated subject failing to maintain at least one of the following response criteria, or downgrading to a lower response criteria according to European Leukemia Net (ELN-2017) is:
a. Complete remission;
b. Complete remission with incomplete hematologic recovery;
c. Morphologic leukemia free state;
d. Partial remission; or
e. Stable Disease lasting 3 months or more.

In one embodiment, the disease progression for a subject suffering from MDS is characterized by the treated subject failing to maintain at least one of the following response criteria, or downgrading to a lower response criteria according to International Working Group (IWG) 2006:
a. Complete remission;
b. Partial remission;
c. Stable disease; or
d. Hematological Improvement.

In one embodiment, the method results in the treated subject achieving at least one of the following response criteria according to European Leukemia Net (ELN-2017):
a. Complete remission;
b. Complete remission with incomplete hematologic recovery;
c. Morphologic leukemia free state;
d. Partial remission; or
e. Stable Disease lasting 3 months or more.

In one embodiment, the method results in the treated subject achieving at least one of the following response criteria according to International Working Group (IWG) 2006:
a. Complete remission;
b. Partial remission;
c. Stable disease; or
d. Hematological Improvement.

In one embodiment, the step of modifying the treatment comprises:
i. a reduction in the total daily dose and/or dosing frequency, wherein the treatment is continued at a relatively lower daily dose and/or a reduced dosing frequency of the compound of formula (I);
ii. a dose hold, wherein the treatment is interrupted until the subject displays an improvement in prohibitive toxicity, and then reinitiating the treatment with the same daily dose and/or dosing frequency of the compound of formula (I); or
iii. a schedule change, wherein the treatment is interrupted until the subject displays improvement in prohibitive toxicity, and then reinitiating the treatment with a relatively lower daily dose and/or a reduced dosing frequency of the compound of formula (I).

In some embodiments, said lower daily dose is a dose selected from three-fourth, half and one-third of the dose at which the prohibitive toxicity is displayed.

In some embodiments, the reduced dosing frequency is half, one-third, or one-fourth of the dosing frequency at which the prohibitive toxicity is displayed.

In accordance with the present disclosure, the step of modifying the treatment as defined herein may be applied more than once, e.g. it may be applied once, twice or three times. In some embodiments, the treatment is terminated in case applying the step of modifying the treatment, optionally several times, does not suffice to remedy the prohibitive toxicity and/or disease progression. In certain preferred embodiments, the step of modifying the treatment, by a reduction in the total daily dose and/or dosing frequency, is done up to 3 times before the treatment is terminated.

In additional embodiment, the step of modifying the treatment comprises escalating, optionally stepwise, such as in one, two, three, four or five steps, the total daily dose and/or dosing frequency of the compound of formula (I), e.g. until the subject displays a prohibitive toxicity. In one embodiment, the escalated total daily dose is up to 1.5 times of the total daily dose at which the disease progression is displayed. In another embodiment, the escalated total daily dose is up to 2-times of the total daily dose at which the disease progression is displayed. In one embodiment, the escalated total daily dose is up to 3-times of the total daily dose at which the disease progression is displayed. In one embodiment, the escalated total daily dose is up to 4-times of the total daily dose at which the disease progression is displayed. In one embodiment, the escalation of the total daily dose and/or dosing frequency is done up to 3 times before the treatment is terminated.

As defined herein before, the method of the present disclosure further comprises the administration of Azacitidine to the subject.

In accordance with the present disclosure, the method typically comprises the administration of Azacitidine or a pharmaceutically acceptable salt thereof via the oral or the parenteral route. In preferred embodiments, Azacitidine is administered to the subject via the parenteral route, preferably via a subcutaneous injection or an intravenous infusion.

In accordance with preferred embodiments of the present disclosure, the method comprises the oral administration of azacitidine or a pharmaceutically acceptable salt thereof in a total daily dose of at least about 25 mg, more preferably at least about 50 mg, at least about 75 mg, at least about 100 mg, at least about 125 mg, at least about 150 mg, at least about 175 mg or at least about 200 mg. In accordance with certain embodiments, the method comprises the oral administration of azacitidine or a pharmaceutically acceptable salt thereof in a total daily dose of about 500 mg or less, more preferably about 450 mg or less, about 400 mg or less, about 350 mg or less, about 325 mg or less or about 300 mg or less. In accordance with embodiments of the present disclosure, the method comprises the oral administration of azacitidine or a pharmaceutically acceptable salt thereof in a total daily dose of about 100-400 mg, more preferably about 150-350 mg, most preferably in about 200-300 mg, e.g. in a total daily dose of about 200 mg or in a total daily dose of about 300 mg. In accordance with other embodiments of the present disclosure, the method comprises the oral administration of azacitidine in a total daily dose of at least about 25 mg, more preferably at least about 50 mg, at least about 75 mg, at least about 100 mg, at least about 125 mg, at least about 150 mg, at least about 175 mg or at least about 200 mg or the oral administration of a pharmaceutically acceptable salt of azacitidine in the equimolar or equipotent dose. In accordance with certain embodiments, the method comprises the oral administration of azacitidine in a total daily dose of about 500 mg or less, more preferably about 450 mg or less, about 400 mg or less, about 350 mg or less, about 325 mg or less or about 300 mg or less, or the oral administration of a pharmaceutically acceptable salt of Azacitidine in the equimolar or equipotent dose. In accordance with embodiments of the present disclosure, the method comprises the oral administration of azacitidine in a total daily dose of about 100-400 mg, more preferably about 150-350 mg, most preferably in about 200-300 mg, e.g. in a total daily dose of about 200 mg or in a total daily dose of about 300 mg, or the oral administration of a pharmaceutically acceptable salt of Azacitidine in the equimolar or equipotent dose.

In one embodiment, the azacitidine or pharmaceutically acceptable salt is administered orally, preferably at the daily doses defined herein above, once daily, for 14 days in a 28 days of treatment cycle for a minimum of 3 to 4 treatment cycles. In some embodiments, Azacitidine is administered at a dose of 300 mg orally once daily, for 14 days in a 28 days of treatment cycle for a minimum of 3 to 4 treatment cycles.

In preferred embodiments of the present disclosure, azacitidine or a pharmaceutically acceptable salt is orally administered to the subject daily, typically at the total daily doses defined here above, for 7 days, followed by a 21 day rest period, during which rest period the subject is not administered azacitidine or a pharmaceutically acceptable salt thereof. In preferred embodiments of the present disclosure, the method comprises a number of such 28-days of treatment cycles, e.g. at least 4 treatment cycles, such as 4 treatment cycles, 5 treatment cycles or 6 treatment cycles.

In preferred embodiments of the present disclosure, a therapeutically effective dose of Azacitidine is administered orally once daily, for 14 days in a 28 days of treatment cycle for a minimum of 3 to 4 treatment cycles.

In other preferred embodiments of the present disclosure, the method comprises the parenteral administration, preferably the administration by subcutaneous injection or intravenous infusion, of azacitidine or a pharmaceutically acceptable salt thereof in a total daily dose of at least about 10 mg/m² of body surface area, more preferably at least about 25 mg/m², at least about 40 mg/m², at least about 45 mg/m², at least about 50 mg/m², at least about 55 mg/m², at least about 60 mg/mg², at least about 65 mg/mg² or at least about 70 mg/mg² e.g. about 75 mg/m². In accordance with certain embodiments, the method comprises the parenteral administration, preferably the administration by subcutaneous injection or intravenous infusion, of azacitidine or a pharmaceutically acceptable salt thereof in a total daily dose of about 200 mg/m² of body surface area or less, more preferably about 150 mg/m² or less, about 125 mg/m² or less, about 100 mg/m² or less, about 90 mg/m² or less, or about 80 mg/m² or less. In accordance with embodiments of the present disclosure, the method comprises the parenteral administration, preferably the administration by subcutaneous injection or intravenous infusion, of azacitidine or a pharmaceutically acceptable salt thereof in a total daily dose of about 25-150 mg/m² of body surface area, more preferably about 50-100 mg/m², most preferably about 70-80 mg/m², e.g. in a total daily dose of about 75 mg/m². In accordance with other embodiments of the present disclosure, the method comprises the parenteral administration, preferably the administration by subcutaneous injection or intravenous infusion, of azacitidine in a total daily dose of at least about 10 mg/m² of body surface area, more preferably at least about 25 mg/m², at least about 40 mg/m², at least about 45 mg/m², at least about 50 mg/m², at least about 55 mg/m², at least about 60 mg/mg², at least about 65 mg/mg² or at least about 70 mg/mg² e.g. about 75 mg/m² or the parenteral administration of a pharmaceutically acceptable salt of azacitidine in the equimolar or equipotent dose. In accordance with certain embodiments, the method comprises the parenteral administration, preferably the administration by subcutaneous injection or intravenous infusion, of azacitidine in a total daily dose of about 200 mg/m² of body surface area or less, more preferably about 150 mg/m² or less, about 125 mg/m² or less, about 100 mg/m² or less, about 90 mg/m² or less, or about 80 mg/m² or less, or the parenteral administration of a pharmaceutically acceptable salt of azacitidine in the equimolar or equipotent dose. In accordance with embodiments of the present disclosure, the method comprises the parenteral administration, preferably the administration by subcutaneous injection or intravenous infusion, of azacitidine in a total daily dose of about 25-150 mg/m² of body surface area, more preferably about 50-100 mg/m², most preferably about 70-80 mg/m², e.g. in a total daily dose of about 75 mg/m², or the parenteral administration of a pharmaceutically acceptable salt of azacitidine in the equimolar or equipotent dose.

In preferred embodiments of the present disclosure, azacitidine or a pharmaceutically acceptable salt thereof is parenterally administered to the subject, preferably as a subcutaneous injection or intravenous infusion, daily, typically at the total daily doses defined here above, for 7 days, followed by a 21 day rest period, during which the subject is not administered azacitidine or a pharmaceutically acceptable salt thereof. In other embodiments of the present disclosure, azacitidine or a pharmaceutically acceptable salt is parenterally administered to the subject, preferably as a subcutaneous injection or intravenous infusion, daily, typically at the total daily doses defined here above, only on days 1-5 and on day 8 and 9 of the 28 days of treatment cycle. In one embodiment, azacitidine or a pharmaceutically acceptable salt thereof is subcutaneously (SC) administered on days 1-5 followed by 2 days rest period, and then two days again in a 28 days of treatment cycle. In another embodiment, azacitidine or a pharmaceutically acceptable salt thereof is intravenously (IV) administered on days 1-5 followed by 2 days off, and then two days again in a 28 days of treatment cycle.

In certain preferred embodiments of the present disclosure, azacitidine or a pharmaceutically acceptable salt thereof is administered as subcutaneous injection or intravenous infusion, daily for 7 days in a 28 days of treatment cycle for a minimum of 4 to 6 treatment cycles. In certain preferred embodiments of the present disclosure, azacitidine or a pharmaceutically acceptable salt thereof is subcutaneously (SC) or intravenously (IV) administered on days 1-5 followed by 2 days off, and then two days again in a 28 days of treatment cycle.

In preferred embodiments of the present disclosure, the method comprises a number of such 28-day treatment cycles, e.g. at least 4 of such 28-day treatment cycles, such as 4 of said treatment cycles, 5 of said treatment cycles or 6 of said treatment cycles.

In one embodiment, the CD47-SIRPα blocking agent and azacitidine are administered simultaneously or sequentially. In certain embodiment of the present disclosure, the treatment with the CD47-SIRPα blocking agent and the treatment with azacitidine or a pharmaceutically acceptable salt thereof, are both started on the same day. In certain embodiment of the present disclosure, the treatment with the CD47-SIRPα blocking agent is started on the day a new 28-day treatment cycle with azacitidine pharmaceutically acceptable salt thereof is started.

### Kits of Parts

Another aspect of the present disclosure is directed to a pharmaceutical kit comprising a package containing a plurality of unit dosage forms and package and/or package insert, wherein said unit dosage form is a unit dosage form comprising a CD47-SIRPα blocking agent selected from the group consisting of the compound of formula (I), stereoisomers thereof, and pharmaceutically acceptable salts, solvates, amides and esters of the compound of formula (I) and stereoisomer thereof, such as the unit dosage forms described herein elsewhere, and wherein said package and/or package insert contains printed instructions to repeatedly administer or self-administer, typically via the oral route, said unit dosage forms in order to accomplish any of the therapeutic objectives as defined herein, such as to treat and/or delay the progression of a disease or a disorder mediated by CD47-SIRPα pathway in a human subject.

In accordance with embodiments of the present disclosure, the pharmaceutical kit preferably comprises a container, such as a cardboard box, holding one or more blister packs, said one or more blister packs containing a plurality of solid unit dosage forms comprising the CD47-SIRPα blocking agent, such as the unit dosage forms described herein elsewhere, preferably a plurality of tablets as defined herein before. In particularly preferred embodiments of the present disclosure, the pharmaceutical kit comprises at least 5, at least 8, at least 10, at least 12 of at least 15 of said unit dosage forms, e.g. 4, 5, 6, 7, 8, 9, 10, 11, 12, 13, 14, 15, 16, 17, 18, 19 or 20 of said unit dosage forms. In one embodiment of the present disclosure, the pharmaceutical kit only comprises unit dosage forms as defined herein that contain the CD47-SIRPα blocking agent as the sole active ingredient. In one embodiment of the present disclosure, the pharmaceutical kit only comprises a plurality of unit dosage forms as defined herein that contain CD47-SIRPα blocking agent as the sole active ingredient and a plurality of unit dosage forms that contain azacitidine or a pharmaceutically acceptable salt thereof as the sole active ingredient, preferably a solid unit dosage form suitable for oral administration or a unit dosage form adapted for parenteral administration, such as a vial containing a powder for reconstitution, typically in the dose amounts recited herein elsewhere. In one embodiment of the present disclosure, the pharmaceutical kit can comprise a plurality of unit dosage forms as defined herein, comprising a CD47-SIRPα blocking agent as well as azacitidine or a pharmaceutically acceptable salt thereof.

In accordance with preferred embodiments of the present disclosure, the pharmaceutical kit comprises a package insert, such as a leaflet inserted into the container, typically a patient information leaflet, containing instructions printed thereon, which information may include a description of the form and composition of the unit dosage forms contained in the kit, an indication of the therapeutic indications for which the product is intended, instructions as to how the product is to be used and information and warnings concerning adverse effects and contraindications associated with the use. It will be understood by those of average skill in the art, based on the information presented herein, that the package insert, such as the leaflet, that is part of the kit according to the present disclosure, will typically contain instructions providing for the therapeutic indications, uses, target subjects, and treatment regimens, as described here above in relation to the methods of treatment of the present disclosure.

### Miscellaneous

As used herein, the term “comprise” and variations of the term, such as “comprising” and “comprises,” are not intended to exclude other additives, components, integers or steps.

The terms “a,” “an,” and “the” and similar referents used herein are to be construed to cover both the singular and the plural unless their usage in context indicates otherwise. Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the phrase “at least one of” preceding a series of items, with the term “and” or “or” to separate any of the items, modifies the list as a whole, rather than each member of the list (i.e., each item).

The phrase “at least one of” does not require selection of at least one of each item listed; rather, the phrase allows a meaning that includes at least one of any one of the items, and/or at least one of any combination of the items, and/or at least one of each of the items. By way of example, the phrase “at least one of A, B, and C” refers to only A, only B, or only C; any combination of A, B, and C; and/or at least one of each of A, B, and C.

The term “about”, whenever used in conjunction with a number or value, generally means within the tolerance range of the equipment used to determine the value, or in some examples, means plus or minus 10%, or plus or minus 5%, or plus or minus 1%, unless otherwise expressly specified. Further, herein the term “substantially” as used herein means a majority, or almost all, or all, or an amount with a range of about 51% to about 100%, for example.

Throughout this text, the use of words in brackets as part of a given term usually means that the wording within brackets specifies a possible option or a possible meaning and that each option or meaning is encompassed by the term.

All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present invention.

The foregoing description is provided to enable a person skilled in the art to practice the various embodiments described herein. While the subject technology has been particularly described with reference to the various embodiments, it should be understood that these are for illustration purposes only and should not be taken as limiting the scope of the subject technology. Those skilled in the art will recognize or be able to ascertain using no more than routine experimentation, many equivalents to the specific embodiments of the invention described herein.

The various aspects and embodiments as defined in the foregoing will be further illustrated by means of the following non-limiting examples.

### EXAMPLES

### Example 1: A Phase I/IIa, Open Label, Dose-Escalation, Study Evaluating the Safety of Compound of Formula (I) in Subjects with Relapsed Advanced Malignancies

Study objective: To evaluate the overall safety profile of the compound of formula (I) in human subjects with relapsed advanced malignancies.

The following table summarizes the (final) study protocol.

TABLE 1
Study Design

| | |
|---|---|
| Inclusion | 1) Male or female human subjects aged ≥18 years. |
| Criteria | 2) Pathological diagnosis of a solid tumor, no existing standard curative or |
| | life prolonging measures, subjects have received at least 2 lines of therapy |
| | in the metastatic setting. |
| | 3) Eastern Cooperative Oncology Group (ECOG) Performance status of 0 |
| | or 1 or 2 (Subjects with disease related ECOG 2 are allowed if deemed by |
| | the Principal Investigator (PI) as related to disease status) |
| | 4) Bone marrow profile: |
| | Absolute Neutrophil Count (ANC) ≥ 1500/μL (without WBC |
| | growth factor support). |
| | Platelet count ≥ 100,000/μL without transfusion support. |
| | Hemoglobin ≥ 9 g/dL (Transfusion allowed to achieve this Hb). |
| | Acceptable organ function as described below: |
| | Total Bilirubin ≤ 1.5 × ULN; (for subjects with known |
| | Gilbert's syndrome a Total Bilirubin ≤ 2.5 × ULN). |
| | AST (SGOT) ≤ 3 × ULN (≤5 × ULN if known liver metastases). |
| | ALT (SGPT) ≤ 3 × ULN (≤5 × ULN if known liver metastases). |
| | Creatinine clearance (CrCl) ≥ 60 mL/min (Cockcroft-Gault |
| | formula for measuring creatinine clearance [eCrCl]: eCrCl = |
| | [140 − Age] × Weight [kg] × [0.85 if Female] / [72 × serum |
| | creatinine (mg/dL)]). |
| | Albumin ≥ 3.0 g/dL. |
| | 5) Ability to swallow and retain oral medications. |
| | 6) Negative serum pregnancy test in women of childbearing potential |
| | (WOCBP). |
| | 7) Women of childbearing potential and men who partner with such a |
| | woman of childbearing potential must agree to use one or more of highly |
| | effective method(s) for contraception for the duration of the study, i.e., |
| | through 28-day follow up visit, after discontinuation of study drug(s). |
| | 8) Evidence of measurable disease per RECIST, v1.1 for solid tumors |
| | (Eisenhauer et al. 2009). Measurable disease for solid tumors is defined as |
| | at least one lesion that can be accurately measured in at least 1 dimension |
| | with a minimum size of 10 mm for non-nodal lesions or 15 mm in short |
| | axis for nodal lesions. For malignant lymphomas, measurable disease is |
| | defined as a lesion that can be accurately measured with a minimum size of |
| | 10 mm in both dimensions or 15 mm in greatest transverse diameter. |
| | AML/MDS subjects are per WHO 2016 criterion. |
| Exclusion | 1) Systemic anti-cancer therapy, such as chemotherapy, or biological |
| Criteria | therapy, immunomodulatory drug therapy, received within the past 28 days |
| | or 5 half-lives, whichever is shorter, from the Cycle 1 Day 1 of the study, |
| | concomitant use of prednisone or medroxyprogesterone allowed, subjects |
| | with CRPC (castrate resistant prostate cancer) continue to receive ongoing |
| | medical castration with LHRH analogues |
| | 2) Subjects eligible for intensive chemotherapy for AML (such as the 3 + 7 |
| | regimen, i.e. a combination of intravenous chemotherapy that includes 7 |
| | days of cytarabine (AraC; 100 mg/m2 per day by continuous infusion) and |
| | 3 days of daunorubicin (DNR; 45 mg/m2 per day, given on days 1, 2, and 3). |
| | 3) CML in blast crisis (i.e., subjects with known bcr-abl positive disease) |
| | 4) Presence of an acute or chronic toxicity resulting from prior anticancer |
| | treatment, with the exception of alopecia or nail changes, that has not |
| | resolved to Grade ≤1, as determined by NCI CTCAE v 5.0 |
| | 5) Definitive Radiotherapy within the last 21 days of Cycle 1 Day 1 (limited |
| | field palliative radiation is allowed with no restrictions during the screening |
| | period or during the trial) |
| | 6) Use of any investigational agent within 28 days or 5 half-lives |
| | (whichever is shorter) prior to Cycle 1 Day 1 |
| | 7) Known symptomatic or untreated or recently treated (≤6 months of |
| | screening) central nervous system (CNS) metastases or CNS leukemia, |
| | subjects with previously treated (>6 months of screening) CNS metastases |
| | or CNS leukemia and are now stable and asymptomatic, from CNS |
| | perspective is allowed |
| | 8) Major surgery ≤ 28 days from Cycle 1 Day 1 (major surgery is defined |
| | as a procedure requiring general anesthesia) |
| | 9) Known to be human immunodeficiency virus (HIV) positive or have an |
| | acquired immunodeficiency syndrome-related illness. |
| | 10) Known active or chronic hepatitis B or hepatitis C infection |
| | 11) Uncontrolled congestive heart failure (New York Heart Association |
| | [NYHA] Class 2-4), angina, myocardial infarction, cerebrovascular |
| | accident, coronary/peripheral artery bypass graft surgery, or transient |
| | ischemic attack, or pulmonary embolism within 3 months prior to Cycle 1 |
| | Day 1 |
| | 12) Ongoing cardiac dysrhythmias requiring treatment of any grade or |
| | treatment of cardiac dysrhythmias in past 3 months, before Cycle 1 Day 1. |
| | 13) The QTcF (corrected QT interval Fridericia method) value in the |
| | screening ECG > 450 ms in males and > 460 ms in females |
| | 14) Previous allogeneic stem cell or bone marrow transplantation |
| | 15) Previous or concomitant additional malignancy, except for basal-cell or |
| | squamous cell carcinoma of the skin or carcinoma in-situ of the uterine |
| | cervix; subjects with other malignancies are eligible if they have remained |
| | disease free for at least 2 years prior to trial entry and in the opinion of the |
| | investigator deemed to have a low likelihood of recurrence |
| | 16) Pregnant or lactating women |
| | 17) Any clinically significant medical, psychiatric or social condition; or |
| | laboratory abnormality that can increase the risk of trial participation or can |
| | interfere with the informed consent process and/or with compliance with |
| | the requirements of the trial or may interfere with the interpretation of the |
| | trial results and, in the Investigator's opinion, can render the subject |
| | inappropriate for entry into trial. |
| | 18) Previously treated with hypomethylating agent(s). |
| Test Product, | Calcium salt of the compound of formula (I) formulated as 25 mg and 100 |
| Dose and | mg capsules for oral administration. |
| Mode of | Dose-cohorts: |
| Administration | Cohort 1 = 25 mg BID (n = 3) |
| | Cohort 2 = 50 mg BID (n = 3) |
| | Cohort 3 = 100 mg BID (n = 7) |
| | Cohort 4 = 200 mg BID (n = 4) and |
| | Cohort 5 = 400 mg BID (n = 3) |
| Duration of | Up to a maximum of 4 cycles of 28 days each |
| Treatment | |
| Dose | In a classic 3 + 3 design with sequential cohorts of three subjects |
| Escalation | treated with incrementally higher doses to evaluate the Dose |
| | Limiting Toxicities (DLTs) for each cohort. |
| | Cycle 1 (28 days) - “DLT Evaluation Period”: Subjects within a |
| | dose cohort are allowed to be enrolled without a waiting period |
| | between subjects, except in Cohort 1. In Cohort 1, there is at least 3 |
| | days of interval between the initiation of the dosing in 1st subject |
| | and the 2nd subject. |
| | Escalation to a higher dose level not allowed to occur until all |
| | subjects within the current dose cohort undergo full safety |
| | assessments through the “DLT Evaluation Period”. |
| | If 1 of 3 subjects in a cohort experiences a DLT during the first |
| | cycle, up to 3 additional subjects are treated at that dose level. |
| | If all subjects in the expanded cohort (6 subjects) undergo full |
| | safety assessments with no additional DLTs observed, i.e., 1/6 |
| | subjects experiencing a DLT, then the dose is escalated in a new |
| | cohort of 3 subjects. |
| | If 2 or more of 3-6 subjects at a given dose level experience a |
| | DLT during the first cycle, then dose cohort is assessed as |
| | having exceeded acceptable safety and up to a total of 6 subjects |
| | are treated at a lower dose level. |
| | If 0 or 1 of 6 subjects experiences a DLT at a dose (and the |
| | higher dose lead to at least 2 DLTs out of 3-6 subjects), then the |
| | dose at which ≤1 of 6 subjects experience a DLT during Cycle |
| | 1 (28 days of dosing) with the next higher dose having at least 2 |
| | of 3-6 subjects experiencing a DLT during Cycle 1 (28 days of |
| | dosing) is determined as the maximum safe dose (MSD). |
| | Safety and tolerability is assessed by the incidence and severity of adverse |
| | events as determined by NCI Common Terminology Criteria for Adverse |
| | Events (NCI CTCAE v 5.0). A Safety Review Committee (SRC) |
| | comprising of the Medical Monitor(s), Principal Investigators (PI) and |
| | Sponsor representatives, reviews safety information and decides upon dose |
| | escalation and further subject enrolment. |
| | For the purpose of this study, a DLT is defined as any of the following |
| | toxicities occurring during Cycle 1, unless clearly and incontrovertibly |
| | relate to underlying malignancy, any other co-morbidities or concomitant |
| | medications or any other extraneous cause” by investigator / SRC: |
| | Treatment related death. |
| | Neutropenia (Absolute Neutrophil Count (ANC) < 1000 /μL) |
| | associated with a single temperature of ≥38.3° C. or two |
| | temperatures of 38° C.-38.2° C. sustained for 1 hour. |
| | Grade 4 neutropenia (ANC < 500/μL), lasting at least 3 days. |
| | Grade 4 thrombocytopenia (Platelet < 25,000/μL), lasting at least 3 |
| | days. |
| | Grade 3 / 4 thrombocytopenia (Platelet < 50,000/ μL) with clinically |
| | significant bleeding |
| | Grade 4 anemia (life threatening and/or with need for urgent |
| | interventions) not due to underlying disease |
| | Positive for Hy's law (AST or ALT ≥ 3 × ULN with concomitant |
| | Total Bilirubin ≥ 2 × ULN, without findings of cholestasis on |
| | radiological scans and no other reasons for increase in AST/ALT |
| | and bilirubin, such as viral hepatitis or acute liver disease or another |
| | drug which can clearly result into these findings). |
| | Any other Grade 3 or higher clinically significant toxicity (except |
| | alopecia or nail changes), per NCI CTCAE v 5. considered as “not |
| | clearly or incontrovertibly related to the underlying malignancy, |
| | any other co-morbidities or concomitant medications or extraneous |
| | causes” by the investigator/SRC. (Grade 3 electrolyte |
| | abnormalities, ALT, AST or bilirubin are excluded from DLT |
| | definition, if these abnormalities resolve to Grade 1 or less within |
| | 72 hours) |
| | Any other toxicity which in the judgment of the Safety Review |
| | Committee was determined to be a DLT. |
| End Points | Primary |
| | Overall safety profile of subjects |
| | Secondary |
| | Adverse Events as characterized by type, frequency, severity (as |
| | graded by National Cancer Institute Common Terminology Criteria |
| | for Adverse Events (NCI CTCAE Version 5.0), timing, seriousness, |
| | and relationship to Test Product. |
| | Laboratory abnormalities as characterized by type, frequency, |
| | severity (as graded by NCI CTCAE Version 5.0) and timing. |
| | Pharmacodynamics (PD) Biomarkers: |
| | Chemokines (MCP-3, MCP-1, MIP-1α & MIP-1β) and Cytokines |
| | (IL-12p40, IL-1A, IL-8, TNFα & IL-6) |
| Schedule of | See Table 2 below |
| Events | |
| Statistical | General: |
| Analysis | Descriptive statistics |
| | Safety Analyses: |
| | Safety observations and measurements include drug exposure, AEs, |
| | safety laboratory tests, vital signs, physical examinations, ECGs, |
| | and ECOG performance status |
| | Coding of Adverse events using the Medical Dictionary for |
| | Regulatory Activities (MedDRA ™) dictionary |
| | Tabulation of the number and percentages of subjects experiencing |
| | AEs by system organ class (SOC), referred term, maximum |
| | severity, and relationship to study drug(s) |
| | Grading of severity of AEs according to the NCI CTCAE v 5.0 |
| | Summary of the number of subjects with dose |
| | reductions/interruptions, SAEs, AEs resulting in trial |
| | discontinuation, and deaths presented by dose, tumor type, and |
| | overall summary of laboratory parameters using descriptive |
| | statistics, by post-dosing shifts in NCI CTCAE toxicity grade |
| | relative to baseline and data listings of clinically significant |
| | abnormalities |
| | Summary of vital signs and ECG data by changes from baseline |
| | values using descriptive statistics (number of subjects, mean, |
| | median, standard deviation, minimum, and maximum); and |
| | physical examination abnormalities |
| | ECOG performance status using frequencies and percentages |

TABLE 2
Schedule of Events

| | | Study Visits | | | | | | | |
|---|---|---|---|---|---|---|---|---|---|
| | | | | | | | | | 28-day |
| | | | | | | | | | follow- |
| | | | | | | | | End of | up visit |
| | | | | | | | | Treatment | Day 28 |
| | | | | | | | | Within 7 | after |
| | Screenings | Cycle 1 | | | | Cycle 2 onwards ^{a} | | days after | last |
| Trial Procedures | (-21 days) | Day 1 | Day 8 | Day 15 | Day 22 | Day 1 | Day15 | last dose | dose |
| Window period | | | | | | ±2 | ±2 | | ±2 |
| (Days) | | | | | | | | | |
| Informed consent | X | | | | | | | | |
| Inclusion/exclusion | X | | | | | | | | |
| criteria | | | | | | | | | |
| Demographics and | X | | | | | | | | |
| Medical history^{b} | | | | | | | | | |
| Physical Exam | X^{c} | X^{d,e} | X^{d} | X^{d} | X^{d} | X^{d} | X^{d} | X^{d} | X^{c} |
| Weight | X | X^{e} | | | | X | | X | X |
| Height | X | | | | | | | | |
| Vital signs^{f} | X | X^{f} | X | X | X | X | X | X | X |
| Hematology^{g} | X | X^{e} | X | X | X | X | X | X | |
| Monitoring for | X | Cycle | Cycle | | | | | | |
| hemolytic anemia^{$} | | 1 Day | 1 Day | | | | | | |
| | | 1, 2 | 8^{$} | | | | | | |
| | | and 5^{$} | | | | | | | |
| Serum chemistry^{g} | X | X^{e} | X | X | X | X | X | X | |
| Coagulation | X | X^{e} | X | X | X | X | | X | |
| parameters^{g} | | | | | | | | | |
| Urinalysis^{g} | X | X^{e} | | X | | X | | X | |
| Electrocardiogram^{h} | X | X | X | X | X | X | | X | |
| Pregnancy testⁱ | X | X | | | | X | | X | |
| ECOG Performance | X | | | | | X | | X | |
| Status | | | | | | | | | |
| Dosing of Calcium | | administered/taken twice every day through all cyclese | | | | | | | |
| salt of compound of | | | | | | | | | |
| formula (I) ^{j} | | | | | | | | | |
| AEs assessment ^{k} | X | X | X | X | X | X | X | X | X |

^{a}After Cycle 1, clinical laboratory tests can be done two days prior to the scheduled visit
^{b}Details of histopathological diagnosis of cancer, all previous treatments and any biomarkers done previously
^{c}Complete physical examination
^{d}Symptom-directed examination
^{e}Cycle 1 Day 1 procedures before test drug administration
^{f}Temperature, pulse rate, blood pressure (seated 5 minutes) pre-dose at all applicable visits; and at five additional times (15, 30 min, 1, 2, and 4 hours (±5 minutes) post-dose) on Cycle 1 Day 1.
^{g}Hematology: complete blood count with differential, platelet count. Reticulocyte count Serum Chemistry: sodium, potassium, chloride, bicarbonate, calcium, phosphorus, magnesium, blood urea nitrogen, creatinine, glucose, bilirubin (direct and total), AST, ALT, alkaline phosphatase, albumin, total protein, LDH, haptoglobin. CPK measurement during Screening and then Day 1 of each cycle. Coagulation Parameters: partial thromboplastic time or activated partial thromboplastin time, and prothrombin time or international normalized ratio. PT/PTT (aPTT) Urinalysis: protein, glucose, ketones, urobilinogen, occult blood, and, when indicated by dipstick abnormality, microscopic sediment evaluation
^{$}Monitoring for hemolytic anemia on Days 1, 2, 5 and 8 of cycle 1 by monitoring CBC, Reticulocyte Count, LDH, Bilirubin (Total, Direct and Indirect), Haptoglobin and urinalysis.
^{h}ECG (after supine/reclined for 5 minutes) pre-dose and 1 hour (±10 min) after the first dose of study drug on Cycle 1 Day 1; ECG pre-dose on Cycle 1 Day 8, Cycle 1 Day 15, Cycle 1 Day 22 and Day 1 of all future Cycles; On Cycle 1 Day 15, ECG two hours post dose; On the ECG days, subjects to take the study drug only after coming to the site and after having the ECG; ECG's also to be performed as clinically indicated
ⁱSerum B-HCG at Screening; urine pregnancy test at subsequent visits. Only for Women with Childbearing Potential. Cycle 1 Day 1 procedures before test drug administration
^{j} Calcium salt of compound of formula (I) on all days through all the cycles as capsules taken orally with water at approXimately the same time on each dosing day; subjects not to eat (drink water) for one hour before and one hour after taking the capsules
^{k} AEs assessment at every timepoint, when study staff interact with the subjects, treatment-related AEs/SAEs ongoing at the time of follow-up visit (28-day after last study drug administration) to be followed up until resolution or return to baseline or until ≤ Grade 1

### Results

### Safety

In all subjects, across dose levels, treatment was well tolerated without any DLT (Dose Limiting Toxicity) or any other safety concerns. No dose limiting toxicities or serious adverse effects, especially hemolytic anemia, were reported in any of the subjects. These findings were quite surprising, given to the ubiquitous expression of CD47 across cell types in human body, especially hematopoietic cells, and its crucial role in multiple fundamental cellular functions, including determining life-span of erythrocytes, cell migration, apoptosis, and axon development, which has impeded the development of small molecule CD47 antagonists as therapeutic options for cancers, in particular advance stage metastatic solid cancers and hematological cancers.

### Adverse Events (AE)

All the reported non-serious adverse events were mild to moderate in nature and the causality of most of them was due to the underlying malignancy. There was no pattern in these AEs. Some of the Grade 1 adverse events were itching, body ache, pain in wrist, elbow or shoulder; nausea, vomiting, myalgia, constipation, abdominal pain, diarrhoea, fever and tingling. A few subjects experienced Grade 2 adverse events like dropping of eye lid or constipation. All of the above lower grade AEs were either considered related to underlying cancer or discovered to have alternate cause (e.g. scabies for itching) or resolved while continuing the trial. No pattern of relationship with dose or cohort was found. No hemolytic anemia was reported in any subject.

### Serious Adverse Events (SAE)

No Grade 3 or 4 toxicities were found in any of the cohorts that could be correlated with the Test Product. Two serious adverse events were reported—one in cohort 2 and another in cohort 3, however, both were determined to be a manifestation of the underlying cancer of the subjects and were assessed as ‘Not Related’ by the respective principal investigators (PI) and the study sponsor.

There were no dose reductions due to treatment-related AEs during the reporting period. No Serious Adverse Reactions (SAR's) or Suspected Unexpected Serious Adverse Reactions (SUSAR's) were reported during the reporting period.

### Pharmacodynamics Biomarkers:

Despite certain variations/outliers among subjects and/or over time, a trend towards downregulation of IL-6 and IL-1α; and a trend towards upregulation of IL12p40, TNFα, MCP-1, MIP-1α, MIP-1β, and MCP-3 as a result of treatment was observable in all cohorts.

Chemokines are known for their role in recruitment of macrophages to sites of infection, damage, or tumors for the purpose of phagocytosis and healing. An upregulation of chemokines followed by administration of the compound of formula (I) is indicative of the CD-47 inhibition mediated enhanced phagocytosis. Disruption of the IL-6-STAT3 axis is known to reduce the ability of tumor cells to escape phagocytosis (Jing Chen et al. Oncoimmunology 2019, VOL. 8, NO. 11, e1652540). A drop in IL-6 level is also indicative of macrophage activation.

Overall, it was found that most of the pharmacodynamic biomarkers displayed modulation on the favourable lines indicating enhanced phagocytosis mediated via CD-47 inhibition.

### Conclusion

Compound of formula (I) was shown to be safe and tolerable up to a dose of 400 mg per oral BID, or a total daily dose of 800 mg. PD biomarker analysis indicated favourable modulation of the cytokines and chemokines indicating the efficacy of the compound of formula (I) against cancer. Surprisingly, the hematological safety of the compound of formula (I) was exceptionally high, with no grade 3 or grade 4 adverse events, and no hemolytic anemia in any of the tested subjects.

### Example 2: A Phase IIa, Two-Part, Open Label, Dose Escalation Study to Evaluate the Safety, Efficacy and PK Profile of a Combination of the Compound of Formula (I) and Azacitidine in Human Subjects with Acute Myeloid Leukemia (AML) or Myelodysplastic Syndrome (MDS)

Main study objective: To evaluate the safety and efficacy of a combination of the compound of formula (I) and Azacitidine in human subjects diagnosed with AML or MDS.

Objective of Part A of the study: to evaluate the safety and tolerability of the orally administered compound of formula (I) at different dose levels, when administered along with Azacitidine (AZA) to human subjects diagnosed with AML or MDS.

Objective of Part B of the study: to evaluate the efficacy of the combination of human subjects diagnosed with AML or MDS.

The following table summarizes the (final) study protocol.

TABLE 3
Study Design:
Part A [Dose escalation - combination of Compound of formula (I) Calcium + Azacitidine]
Part B [Randomization - combination of Compound of formula (I) Calcium + Azacitidine]

| | |
|---|---|
| Inclusion | 1) Signed informed consent to comply with all study related activities |
| Criteria | 2) Male or female human subjects aged ≥18 years |
| | 3) Human subjects meeting the following criteria: |
| | Part A & B: |
| | Diagnosis of Acute myeloid leukemia (AML) according to the |
| | World Health Organization (WHO 2016) criteria, OR |
| | Myelodysplastic syndrome (MDS) according to the WHO |
| | classification (WHO 2016). |
| | Part A: |
| | AML: |
| | newly diagnosed AML subjects who are ineligible for |
| | intensive chemotherapy |
| | subjects diagnosed with relapsed / refractory AML |
| | who has received at least one line of previous therapy |
| | other than hypomethylating agents, and eligible for |
| | receiving Azacitidine |
| | OR |
| | MDS: Intermediate / High-Risk / Very High-Risk |
| | Myelodysplastic syndrome (MDS) with IPSS-R score > 3.5, |
| | by IPSS-R criterion and eligible to receive Azacitidine |
| | Part B: |
| | AML: subjects with newly diagnosed AML not eligible for |
| | intensive chemotherapy |
| | MDS: newly diagnosed Intermediate / High-Risk / Very High- |
| | Risk Myelodysplastic syndrome (MDS) with IPSS-R score |
| | 3.5, by IPSS-R criterion and eligible to receive Azacitidine |
| | 4) Bone marrow profile: |
| | WBC < 20,000/μL (Hydroxyurea allowed to reduce WBC |
| | count to <20,000/μl) |
| | Platelet count ≥ 50,000/μL without transfusion support |
| | Hemoglobin (Hb) ≥ 9 g/dL (Transfusion allowed to achieve |
| | this Hb) |
| | 5) Organ function: |
| | Total Bilirubin ≤ 1.5 × ULN; (Subjects with known Gilbert's |
| | syndrome - Total Bilirubin ≤ 2.5 × ULN) |
| | AST (SGOT) ≤ 3 × ULN (≤5 × ULN if known liver |
| | metastases) |
| | ALT (SGPT) ≤ 3 × ULN (≤5 × ULN if known liver |
| | metastases) |
| | Creatinine clearance (CrCl) ≥ 60 mL/min (Cockcroft-Gault |
| | formula for measuring creatinine clearance [eCrCl]: eCrCl = |
| | [140 − Age] × Weight [kg] × [0.85 if Female] / [72 × serum |
| | creatinine (mg/dL)]) |
| | Albumin ≥ 3.0 g/dL |
| Exclusion | 1) Systemic anti-cancer therapy, such as chemotherapy, or biological |
| Criteria | therapy, immunomodulatory drug therapy, received within the past 28 |
| | days or 5 half-lives, whichever is shorter, from the Cycle 1 Day 1 of |
| | the study; concomitant use of prednisone or medroxyprogesterone |
| | allowed; subjects with CRPC (castrate resistant prostate cancer) |
| | continue to receive ongoing medical castration with LHRH analogues |
| | 2) Subjects eligible for intensive chemotherapy for AML (such as the |
| | 3 + 7 regimen, i.e. a combination of intravenous chemotherapy that |
| | includes 7 days of cytarabine (AraC; 100 mg/m2 per day by |
| | continuous infusion) and 3 days of daunorubicin (DNR; 45 mg/m2 per |
| | day, given on days 1, 2, and 3). |
| | 3) CML in blast crisis (i.e., subjects with known bcr-abl positive |
| | disease) |
| | 4) Presence of an acute or chronic toxicity resulting from prior |
| | anticancer treatment, with the exception of alopecia or nail changes |
| | not resolved to Grade ≤1, as determined by NCI CTCAE v 5.0 |
| | 5) Definitive Radiotherapy within the last 21 days of Cycle 1 Day 1; |
| | limited field palliative radiation allowed with no restrictions during |
| | the screening period or during the trial |
| | 6) Use of any investigational agent within 28 days or 5 half-lives |
| | (whichever is shorter) prior to Cycle 1 Day 1 |
| | 7) Known symptomatic or untreated or recently treated (≤6 months of |
| | screening) central nervous system (CNS) metastases or CNS |
| | leukemia; Subjects with previously treated (>6 months of screening) |
| | CNS metastases or CNS leukemia but stable and asymptomatic from |
| | CNS perspective allowed |
| | 8) Major surgery ≤ 28 days from Cycle 1 Day 1 (major surgery is |
| | defined as a procedure requiring general anesthesia) |
| | 9) Known to be human immunodeficiency virus (HIV) positive or |
| | having an acquired immunodeficiency syndrome-related illness |
| | 10) Known active or chronic hepatitis B or hepatitis C infection |
| | 11) Uncontrolled congestive heart failure (New York Heart |
| | Association [NYHA] Class 2-4), angina, myocardial infarction, |
| | cerebrovascular accident, coronary/peripheral artery bypass graft |
| | surgery, or transient ischemic attack, or pulmonary embolism within |
| | 3 months prior to Cycle 1 Day 1 |
| | 12) Ongoing cardiac dysrhythmias requiring treatment of and grade or |
| | treatment of cardiac dysrhythmias in past 3 months, before Cycle 1 |
| | Day 1 |
| | 13) The QTcF (corrected QT interval Fridericia method) value in the |
| | screening ECG > 450 ms in males and >460 ms in females |
| | 14) Previous allogeneic stem cell or bone marrow transplantation |
| | 15) Previous or concomitant additional malignancy, except for basal- |
| | cell or squamous cell carcinoma of the skin or carcinoma in-situ of the |
| | uterine cervix; subjects with other malignancies eligible if they have |
| | remained disease free for at least 2 years prior to trial entry and in the |
| | opinion of the investigator deemed to have a low likelihood of |
| | recurrence |
| | 16) Pregnant or lactating women |
| | 17) Any clinically significant medical, psychiatric or social condition; |
| | or laboratory abnormality that can increase the risk of trial |
| | participation or interfere with the informed consent process and/or |
| | with compliance with the requirements of the trial or may interfere |
| | with the interpretation of the trial results and, in the Investigator's |
| | opinion, make the subject inappropriate for entry into this trial |
| | 18) Previously treated with hypomethylating agent(s) |
| Dose, Dosing | PART A |
| Regimen and | Calcium salt of Compound of formula (I) + Azacitidine (AZA) |
| Duration of | Dose escalation of the compound of formula (I) was done in a 3 + 3 |
| Treatment | manner while keeping the Azacitidine dose as constant. |
| | 3 dose-cohorts: |
| | Cohort 1 = 200 mg BID (n = 6) |
| | Cohort 2 = 300 mg BID (n = 6) |
| | Cohort 3 = 400 mg BID (n = 6) |
| | PART B |
| | Randomization of subjects to two dose levels (n = 45 each) - 200 mg |
| | BID versus 300 mg BID (or 400 mg BID) of compound of formula I + Azacitidine |
| | In both, Part A and B, subjects receive compound of formula (I) orally |
| | (PO) twice daily (BID) on days 1-28 and Azacitidine subcutaneously |
| | (SC) or intravenously (IV) over 15 minutes on days 1-7. PI to decide |
| | if Azacitidine to be given for 5 days followed by 2 days off and then |
| | again for two days |
| | Subject continues to receive study drugs for the entire duration of trial, |
| | until the subject displays progressive disease or prohibitive toxicity. If |
| | compound of formula (I) stopped for any reason, then Azacitidine |
| | must also stop |
| Intra-subject | The study allows an intra-subject dose increase to the next dose level, |
| dose | from Cycle 2 onwards in Part A of the study. Intra-subject dose |
| escalation | escalation is allowed once only if the subject has not suffered a DLT |
| | and the subject tolerates the study drug well in the opinion of the PI. |
| Dose Reductions | For subjects in Part A who develop DLT in cycle 1, the |
| and Holds | compound of formula (I), or the compound of formula (I)/ |
| | Azacitidine to be permanently stopped |
| | If DLT-equivalent event happens in Cycle 2 onwards or during |
| | expansion, then once the event improves to Grade 2, then re- |
| | initiate study drugs at 50% lower dose - in any individual subject, |
| | a maximum of two dose reductions permitted |
| | If any other study drug related AE (non-DLT in Cycle 1 or non- |
| | DLT equivalent in Cycle 2 onwards or expansion cohorts) occur |
| | during the subject's participation in the study, the Investigator to |
| | determine whether the toxicity requires dose hold/reduction or |
| | discontinuation from treatment. |
| | Upon a “Dose-Reduction” in a subject, an increase of the dose |
| | can be done, to the maximum of the original dose |
| | In general, if the dose is held in a subject for 28 days or more, for |
| | any reason, then the subject is withdrawn from the study. |
| | However, if the drug hold is longer than 28 days and there is |
| | evidence of clinical benefit and the Investigator believes it to be |
| | in the best interest of the subject to resume study drug, then the |
| | compound of formula (I) dosing is allowed at the same or the |
| | lower dose (50%) level following discussion with the Sponsor's |
| | Medical Monitor |
| | For Azacitidine: The dosage and administration of Azacitidine is |
| | as per the package insert of the drug (Days 1-7 of 28-day cycle). |
| | However, if the local practices follow 5 days of dosing followed |
| | by 2 days of off followed by another 5 days of dosing, then the |
| | same is also allowed, as per the judgment of the PI |
| | Dose adjustments can be planned based on hematology |
| | laboratory values, serum electrolytes and renal toxicity as |
| | detailed in the prescribing information of Azacitidine |
| | Definition of DLT |
| | PART A and B |
| | Any of the following toxicities occurring during Cycle 1 of Part A, |
| | unless considered clearly and incontrovertibly related to underlying |
| | malignancy, any other co-morbidities or concomitant medications or |
| | any other extraneous cause” by investigator /SRC: |
| | Treatment related death. |
| | Positive for Hy's law (AST or ALT ≥ 3 × ULN with |
| | concomitant Total Bilirubin ≥ 2 × ULN, without findings of |
| | cholestasis on radiological scans and no other reasons for |
| | increase in AST/ALT and bilirubin, such as viral hepatitis or |
| | acute liver disease or another drug which can clearly result into |
| | these findings) |
| | Grade 4 neutropenia (ANC < 500/μL) at Day 42, in the absence |
| | of persistent disease (AML/MDS) |
| | Any other Grade 3 or higher clinically significant toxicity |
| | (except alopecia or nail changes), per NCI CTCAE v 5.0 which |
| | is considered as “not clearly or incontrovertibly related to the |
| | underlying malignancy, any other co-morbidities or |
| | concomitant medications or extraneous causes” by the |
| | investigator/SRC (Grade 3 electrolyte abnormalities, ALT, |
| | AST or bilirubin excluded from DLT definition, if these |
| | abnormalities resolve to Grade 1 or less within 72 hours). |
| | Any other toxicity which in the judgment of the Safety Review |
| | Committee is a DLT. |
| | Note: As per accepted practice, hematological AEs (other than those |
| | mentioned above) are not part of DLT for Part A as these events |
| | happen not uncommonly in AML/MDS. |
| Schedule | See Table 4 |
| of Events | |
| End Points | PART A |
| | Primary |
| | Overall safety profile of the combination of Calcium salt of |
| | compound of formula (I) and Azacitidine in human subjects |
| | with AML or intermediate risk / high-risk/ very high-risk MDS |
| | Secondary |
| | Adverse Events as characterized by type, frequency, severity |
| | (as graded by National Cancer Institute Common Terminology |
| | Criteria for Adverse Events (NCI CTCAE Version 5.0), |
| | timing, seriousness, and relationship to study therapy |
| | Laboratory abnormalities as characterized by type, frequency, |
| | severity (as graded by NCI CTCAE Version 5.0) and timing |
| | Preliminary evidence of efficacy of the combination in AML |
| | or MDS. |
| | For AML subjects (according to ELN 2017 criteria): |
| | Complete remission (CR) |
| | Complete remission with incomplete hematologic |
| | recovery |
| | Morphologic leukemia free state |
| | Partial remission (PR) |
| | Stable Disease lasting 3 months or more |
| | For MDS subjects (according to IWG 2006 criteria) |
| | Complete remission (CR) |
| | Partial remission (PR) |
| | Stable disease |
| | Hematological Improvement |
| | Exploratory |
| | Pharmacodynamics (PD) effects on selected biomarkers. |
| | PART B |
| | Primary |
| | Efficacy of the combination in specific population of AML or |
| | MDS, as assessed by Overall Response Rate (ORR) (according |
| | to ELN 2017 criteria in AML and according to IWG 2006 for |
| | MDS) |
| | Secondary |
| | Additional efficacy parameters of the combination in specific |
| | population of AML or MDS by CR, PR (according to ELN |
| | 2017 in AML and according to IWG 2006 in MDS) |
| | Overall safety profile of the combination as measured by: |
| | Adverse Events as characterized by type, frequency, |
| | severity (as graded by National Cancer Institute |
| | Common Terminology Criteria for Adverse Events |
| | (NCI CTCAE), timing, seriousness, and relationship to |
| | study therapy |
| | Laboratory abnormalities as characterized by type, |
| | frequency, severity (as graded by NCI CTCAE) and |
| | timing |
| | Exploratory |
| | Pharmacodynamics (PD) effects on selected biomarkers |
| Statistical | General: Descriptive statistics |
| Analysis | Safety Analyses: |
| | Safety observations and measurements include drug exposure, |
| | AEs, safety laboratory tests, vital signs, physical examinations, |
| | ECGs, and ECOG performance status |
| | Coding of adverse events using the Medical Dictionary for |
| | Regulatory Activities (MedDRA ™) dictionary |
| | Tabulation of the number and percentages of subjects experiencing |
| | AEs by system organ class (SOC), preferred term, maximum |
| | severity, and relationship to study drug(s) |
| | Grading of the severity of AEs according to the NCI CTCAE v 5.0 |
| | Summaries of the number of subjects with dose |
| | reductions/interruptions, SAEs, AEs resulting in trial |
| | discontinuation, and deaths by dose, tumor type, and overall |
| | Summary of laboratory parameters using descriptive statistics, by |
| | post-dosing shifts in NCI CTCAE toxicity grade relative to |
| | baseline and data listings of clinically significant abnormalities |
| | Summary of vital signs and ECG data by changes from baseline |
| | values using descriptive statistics (number of subjects, mean, |
| | median, standard deviation, minimum, and maximum); and |
| | physical examination abnormalities |
| | ECOG performance status using frequencies and percentages |
| | Efficacy Analyses: |
| | AML - ELN 2017 criteria |
| | MDS - IWG 2006 criteria |
| | Exploratory Biomarkers: Summaries of PD biomarkers by dose |
| | groups |

TABLE 4
Schedule of Events [Part A and B]

| | | Study Visits | | | | | | | |
|---|---|---|---|---|---|---|---|---|---|
| | | | | | | | | | 28-day |
| | | | | | | | | | follow- |
| | | | | | | | | End of | up visit |
| | | | | | | | | Treatment | Day 28 |
| | | Cycle 1 | | | | Cycle 2 onwards ^{a} | | Within 7 | after |
| | Screenings | Day | Day | Day | Day | Day | Day | days after | last |
| Trial Procedures | (−21 days) | 1 | 8 | 15 | 22 | 1 | 15 | last dose | dose |
| Window period | | | | | | ±2 | ±2 | | ±2 |
| (Days) | | | | | | | | | |
| Informed consent | X | | | | | | | | |
| Inclusion/exclusion | X | | | | | | | | |
| criteria | | | | | | | | | |
| Demographics and | X | | | | | | | | |
| Medical history ^{b} | | | | | | | | | |
| Physical Exam | X^{c} | X^{d,e} | X^{d} | X^{d} | X^{d} | X^{d} | X^{d} | X^{d} | X^{c} |
| Weight | X | X^{e} | | | | X | | X | X |
| Height | X | | | | | | | | |
| Vital signs ^{f} | X | X^{f} | X | X | X | X | X | X | X |
| Hematology^{g} | X | X^{e} | X | X | X | X | X | X | |
| Monitoring for | X | Cycle | Cycle | | | | | | |
| hemolytic anemia^{$} | | 1 Day | 1 Day | | | | | | |
| | | 1,2 | 8^{$} | | | | | | |
| | | and 5^{$} | | | | | | | |
| Serum chemistry^{g} | X | X^{e} | X | X | X | X | X | X | |
| Coagulation | X | X^{e} | X | X | X | X | | X | |
| parameters^{g} | | | | | | | | | |
| Urinalysis^{g} | X | X^{e} | | X | | X | | X | |
| Electrocardiogram^{h} | X | X | X | X | X | X | | X | |
| Pregnancy testⁱ | X | X^{e} | | | | X | | X | |
| ECOG Performance | X | X | | | | X | | X | |
| Status | | | | | | | | | |
| Bone marrow | X | Please refer to footnote ‘j’ | | | | | | | |
| assessment for disease | | | | | | | | | |
| evaluation^{j} | | | | | | | | | |
| Dosing of Calcium salt | | administered/taken twice every day through all cycles^{k} | | | | | | | |
| of compound of | | | | | | | | | |
| formula (I) k | | | | | | | | | |
| Azacitidine dosing¹ | | Administered SC or IV over 15 minutes on days 1-7 of each cycle. Subject was | | | | | | | |
| | | admitted, if needed, for logistical reasons on Days 1-7 of each cycle for | | | | | | | |
| | | Azacitidine administration. | | | | | | | |
| 24 hours housing, as | | X | X | X | X | | | | |
| required | | | | | | | | | |
| PD biomarker samples | | X | X | X | | X | | | |
| (blood samples)^{m} | | | | | | | | | |
| Previous/concomitant | X | X | X | X | X | X | X | X | X |
| medications | | | | | | | | | |
| AEs assessment^{a} | X | X | X | X | X | X | X | X | X |

^{a} All assessments prior to treatment, unless specified otherwise in the protocol. After Cycle 1, clinical laboratory tests can be done two days prior to the scheduled visit.
^{b} Collect histopathological diagnosis of cancer, all previous treatments and any biomarkers done previously as part of “Medical History”
^{c}Complete physical examination
^{d}Symptom-directed examination
^{e}All the pre-dose Cycle 1 Day 1 procedures before administration of compound of formula (I), except vital signs, ECG, pre-dose PK and pre-dose PD up to 2 days prior
^{f} Temperature, pulse rate, blood pressure (seated 5 minutes) pre-dose at all applicable visits; 15, 30 min, 1, 2, and 4 hours (±5 minutes) post-dose on Cycle 1 Day 1, vital signs measurement on the days of Azacitidine administration as per institutional practice
^{g}Hematology: complete blood count with differential, platelet count. Reticulocyte count
Serum Chemistry: sodium, potassium, chloride, bicarbonate, calcium, phosphorus, magnesium, blood urea nitrogen, creatinine, glucose, bilirubin (direct and total), AST, ALT,
alkaline phosphatase, albumin, total protein, LDH, Haptoglobin. CPK measurement during Screening and end of treatment visit only
Coagulation Parameters: partial thromboplastic time or activated partial thromboplastin time, and prothrombin time or international normalized ratio. PT/PTT (aPTT)
Urinalysis: protein, glucose, ketones, urobilinogen, occult blood, and, when indicated by dipstick abnormality, microscopic sediment evaluation. Clinical laboratory tests within 48 hours prior to scheduled Day 1 or Day 15 of the respective cycles
^{$}Monitoring for hemolytic anemia and tumor lysis syndrome (TLS) once daily on Days 1, 2, 4, 6 and 8 of cycle 1 by monitoring CBC, Reticulocyte Count, Haptoglobin, LDH, Bilirubin (Total, Indirect and Direct), Uric Acid, Serum Potassium, Serum Calcium, Serum Phosphate, Creatinine, Blood Urea Nitrogen and urinalysis parameters.
^{h}ECG (after supine/reclined for 5 minutes) pre-dose and 1 hour (±10 min) after the first dose of study drug on Cycle 1 Day 1; also pre-dose on Cycle 1 Day 8, day 15, day 22 and then day 1 of each subsequent cycle; on cycle 1 day 15, ECG two-hours post dose, or as clinically indicated
ⁱSerum β-HCG at Screening; urine pregnancy test at subsequent visits. Only for Women with Childbearing Potential.
j) Bone marrow assessment for disease evaluation for subjects of both AML and MDS by bone marrow aspirates and biopsy; Bone marrow assessment at screening (within 28 days of the first dose of study drug); If bone marrow assessment is already done prior to screening, (within 28 days of expected Cycle 1 Day 1), then same can be used for screening; Subsequent bone marrow aspirates collection on Cycle 3 D 1 (±7 days) and every 2 cycles afterwards, and as clinically indicated., such as when clinical progression is suspected. After C5D1 (±7 days) bone marrow evaluation, the frequency of bone marrow assessments can be decreased to every 3 cycles, as per the judgment of PI. However, for documentation of CR or PR or marrow CR, bone marrow assessment is required.
k) Calcium salt of compound of formula (I) on all days through all the cycles taken as capsules orally with water at approximately the same time on each dosing day. Subjects not to eat (drink water) for one hour before and one hour after taking the capsules.
^{l}Azacitidine dosing: Azacitidine administration subcutaneously (SC) or intravenously (IV) over 15 minutes on days 1-7 of the cycle. Local practice patterns (such as 5 days on followed by 2 days off and then 2 days on) can be considered by the PI.
^{m}Pharmacodynamic markers sampling: In Part A, cycle 1, PD samples on Day 1 (Pre-dose, and 12 hours), i.e., before evening dose), Day 8 and Day 15. PD samples pre-dose in Cycle 2 Day 1 and Cycle 3 Day 1.
ⁿAEs were assessment at every timepoint, when study staff interacts with the subject.
Treatment-related AEs/SAEs ongoing at the time of follow-up visit (28-day after last study drug administration) continue to be followed until resolution or return to baseline or until ≤ Grade 1.

### Results

The presence of 20% or more blast cells in blood or bone marrow is a key diagnostic criterion for acute leukemia, including acute myeloid leukemia (AML).

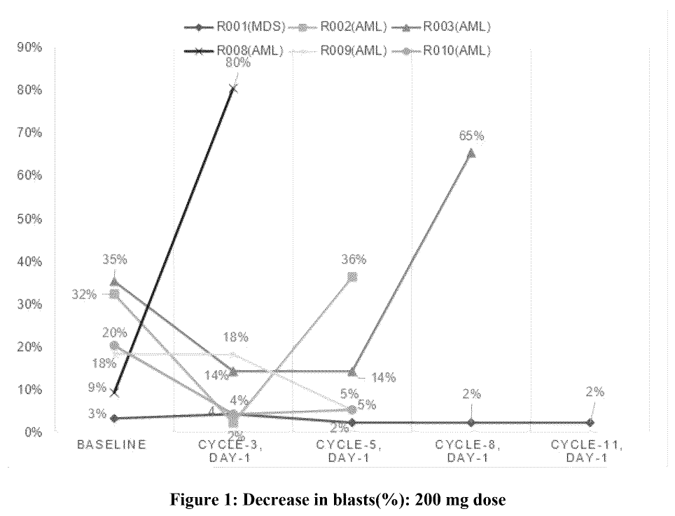

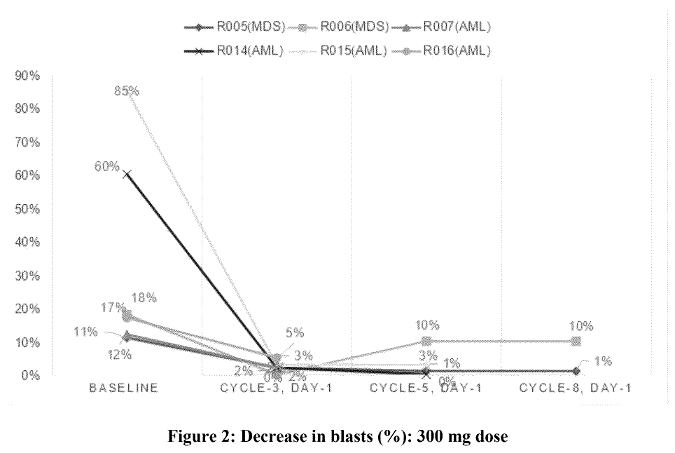

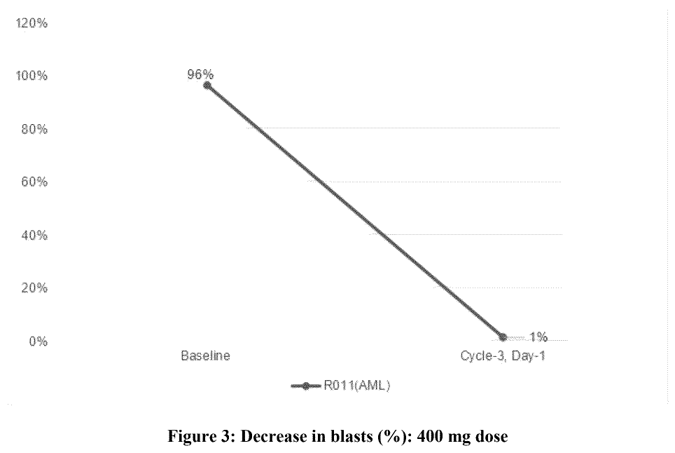

The results are presented in the following table 5 and FIGS. 1-3

TABLE 5

| Dose level | ORR at cycle 3 |
|---|---|
| 200 mg BID | MDS: 0/1 |
| | AML: 40% (2/5) |
| 300 mg BID | MDS: 0/2 (significant blast reduction in both; by criteria |
| | stable disease). |
| | AML: 75% (3/4) |
| 400 mg BID | AML: 50%, (1/2) |

Objective Response as per ELN 2017 criteria:
1) CR (Complete Remission).
2) CRI (Complete Remission with incomplete hematologic recovery).
3) PR (Partial Remission).
4) MLFS (Morphologic Leukemia Free State)

It was observed that the ORR across patients and across tested doses was achieved. It was also observed that at 300 mg BID of compound I and Azacitidine, the efficacy as measured by Blast % change was sustained in all the patients.

### Example 3: In-Vitro Azacytidine Combination Effect on Phagocytosis Correlated with Induction of Calreticulin

The objective of this study was to determine the effect of Calcium salt of compound of formula (I) and azacitidine mediated induction of phagocytosis of HL-60 cells by human macrophages. Different doses of Calcium salt of compound of formula (I) were tested to determine increase in phagocytosis of HL-60 cells (with or without azacitidine treatment) by macrophages isolated from healthy human volunteers.

Preparation of test compound: 20 mM stock concentration of Calcium salt of compound of formula (I) was prepared. The stock was further diluted to get intermediate and final concentrations. The final assay volume was 200 μL. Medium used for dilution is RPMI media. Four concentrations of Calcium salt of compound of formula (I) ranging from 10 μM to 0.3 μM were used in the assay.

Preparation of macrophage culture media: 5 mL of FBS and 10 μL of recombinant human M CSF (100 μg/mL stock) was added to 45 mL of RPMI to prepare macrophage culture media as and when required.

Preparation of HL-60 cell culture media: 10 mL of FBS was added to 40 mL of IMDM media to prepare HL-60 cell culture media.

Preparation of working stock of azacitidine: A 20 mM stock of azacitidine prepared by adding 172.1 μL of DMSO in 0.84 mg of azacitidine. 4.5 μL of 20 mM stock of azacitidine was added to 25.5 μL of DMSO to prepare 3 mM azacitidine working stock solution. This is 1000× of the desired final treatment concentration of 3 μM.

Preparation of working stock of Mouse IgG1 Kappa isotype control: Molarity of mouse IgG1 kappa isotype control antibody was calculated using online molarity calculator (https://www.graphpad.com/quickcalcs/Molarityform.cfm). Formula weight of antibody was 150 KDa. Molarity of isotype control antibody stock was 6.6667 μM. 3 μL of isotype control antibody stock solution was added to 197 μL RPMI media to prepare 100 nM of working stock solution. This is 10× stock of the required conc of isotype control. Preparation of working stock of anti-human CD47 antibody Molarity of anti-human CD47 antibody was calculated using online molarity calculator (https://www.graphpad.com/quickcalcs/Molarityform.cfm). Formula weight of anti-Human CD47 antibody was 150 KDa. Molarity of anti-human CD47 antibody stock was 6.6667 μM. This stock of anti-human CD47 antibody was further diluted to get intermediate and final concentrations as described below. 3 μL of anti-human CD47 antibody stock solution was added to 197 μL RPMI media to prepare 100 nM of working stock solution (10×).

Preparation of test system: 90 mL whole blood from healthy volunteer was collected in a Na-Heparin vacutainer and stored at room temperature until use. PBMCs isolated from blood were used for isolating monocytes and to differentiate them into macrophages for the phagocytosis assay.

### Isolation of Monocytes and Differentiation to Macrophages:

90 mL blood was collected from healthy volunteer in NA-Heparin vacutainer. PBMCs were isolated and were resuspended in 50 mL serum free RPMI 1640 media. For isolation of monocytes, 10 mL of PBMC cell suspension was seeded in each T75 culture flask (total 5 flasks) and were cultured at 37 degree for 2 hours. Monocytes were attached to culture flask by adherence method. Media from these flasks was discarded and attached monocytes were washed once with prewarmed PBS. Monocytes were further cultured in 10 mL of macrophage culture media and were differentiated to macrophages for 10 days. Media from each flask was changed every alternate day or after every 2 days with fresh macrophage culture media.

### Revival, Culturing and Azacitidine Treatment of HL-60 Cells:

A vial of HL-60 cell stock was taken from liquid nitrogen. Cells were thawed in a water bath at 37° C. till the last crystal is thawed. Cells were transferred to 50 mL of culture media in a falcon tube and centrifuged at 800 rpm for 8 minutes. Cells were further cultured in 20 mL of HL-60 cell culture media in T75 flasks at 37° C. at 5% CO2 for 24 hrs. After 24 hrs, the cells were centrifuged at 800 rpm for 5 mins. The cells were cultured in 25 mL of complete media at 37° C. and 5% CO2 for 2 more days. After 2 days, HL-60 cells were cultured in 20 mL of complete media in T75 flasks at 37° C. at 5% CO2 for 3 days. Further, HL-60 cells were treated with azacitidine to induce eat me signals. Cultured cells were washed by centrifugation at 800 rpm for 5 minutes at RT with 20-30 mL of serum free IMDM media. Cells were counted and were resuspended in complete HL-60 culture media to prepare 0.5 million cells per mL concentration. Six million tumor cells prepared in 12 mL of HL-60 culture media were seeded in two T-25 flasks. One of the two flasks was added with 12 μL of 3 mM azacitidine to attain a final concentration of 3 μM. Additionally, other flask of HL-60 cells was added with 12 μL of DMSO. Both the flasks were incubated at 37° C. in the CO2 incubator for 72 hrs.

### Phagocytosis Assay Set Up:

Phagocytosis assay was set up with differentiated human macrophages and HL-60 cells treated with either DMSO or azacitidine. On the day of phagocytosis assay, macrophages were serum starved by replacing media from flasks containing differentiated macrophages with 10 mL of serum free RPMI media for 2 hrs at 37° C. in 5% CO2.

Meanwhile, azacitidine or DMSO treated HL-60 cells were pelleted in separate 15 mL falcon tubes by centrifugation at 1200 rpm for 5 min. Cells were counted and re-suspended at a concentration of 2×106 cells per mL of serum free RPMI media. CFSE staining of azacitidine and DMSO treated tumor cells was performed by adding 5 μL of 100 M stock of CFSE to 2.5 mL of cell suspension. Cells were stained for 10 min at 37° C. in 5% CO2. CFSE staining reaction was stopped by adding 10 mL of chilled DPBS to the stained cells and centrifuged at 1200 rpm to wash the CFSE labelled cells. Washed cells were counted and 5.1×106 HL-60 cells (DMSO or azacitidine treated) were resuspended in 3.4 mL RPMI media. CFSE stained HL-60 cells (DMSO and azacitidine) were seeded at a concentration of 0.15×106 per well of 96 well round bottom plate in 100 μL media. Cells were treated with 10 nM concentration of anti-human CD47 antibody or Mouse IgG1 K Isotype control or various concentrations of Compound (I) calcium (prepared in 20 μL of RPMI media) in 96-well round bottom plate for 30 minutes at 37° C.

The serum starved macrophages were harvested using cell dissociation buffer. 5 mL of cell dissociation buffer was added to each T75 flask containing macrophages and incubated at 37° C. in 5% CO2 for 6 min. After 5 minutes, macrophages that were still attached to the flask and were dislodged by scraping with cell scraper. Macrophages from all the flasks were collected and washed with 15 mL of DPBS by centrifugation at 1200 rpm for 5 min. Macrophages were resuspended in 1 mL of RPMI and counted. Macrophage suspension was adjusted to concentration of 0.625×106 cells per mL using RPMI 1640 media. 80 μL of this macrophage cell suspension was added into respective wells of a 96 well plate with HL-60 cells (co incubated with either of the antibodies or compound) and mixed well. Additional 2 wells were added with 80 μL of macrophage suspension and 120 μL of RPMI 1640 media respectively as controls for flowcytometry analysis. The total volume of phagocytosis assay was 200 μL per well. Phagocytosis was allowed to happen by incubating the plate for 2 hours at 37° C. in 5% CO2. After 2 hours, phagocytosis was stopped by centrifugation of the assay plate at 1200 rpm for 5 min and discarding the supernatant. Cells pelleted at the bottom of each well were stained with 100 μL staining buffer containing 1 μL of anti-human CD11b-APC antibody cocktail for 30 min at 4° C. in dark. 100 μL staining buffer alone was added to unstained control cells. Cells were washed with 100 μL of PBS by centrifugation at 1200 rpm for 5 min and re-suspended in 100 μL of IC fixation buffer for fixation. Fixed cells were stored overnight at 4° C. in dark before acquiring by FACS. Next day, cells were washed with 100 μL PBS by centrifugation at 1200 rpm for 5 min and added with 200 μL of staining buffer to each well. Samples were acquired on FACS Verse instrument.

### Data Analysis:

FCS files were analysed by FlowJo software. Cells positive for both fluorescence of CD11b as well as CFSE were considered positive for phagocytosis. Samples were first gated by plotting forward scatter height and side scatter height. Unstained macrophages and CD11b stained macrophages were used as controls for the instrument settings. Macrophages were defined positive for CD11b-APC. Macrophages were further divided into quadrants based on FITC (CFSE) and APC signal. Cells positive for FITC (CFSE) and APC are considered as phagocytosed by macrophages. Raw data of % phagocytosis was obtained in the form of excel sheet from FlowJo. % Phagocytosis from HL-60 cells treated with isotype control antibody (background phagocytosis) was subtracted from % phagocytosis of HL-60 cells treated with compound or anti-human CD47 antibody to get corrected phagocytosis values. Corrected phagocytosis values were further normalized with phagocytosis in presence of 10 nM of anti-human CD47 antibody (B6H12) using the formula % Normalized phagocytosis=[(corrected % phagocytosis in presence of test item or antibody)/(corrected phagocytosis in presence of 10 nM of anti-human CD47)*100]% Normalized phagocytosis in treated samples will be plotted against the respective concentrations using GraphPad prism 9.1.1.

### Results and Discussion:

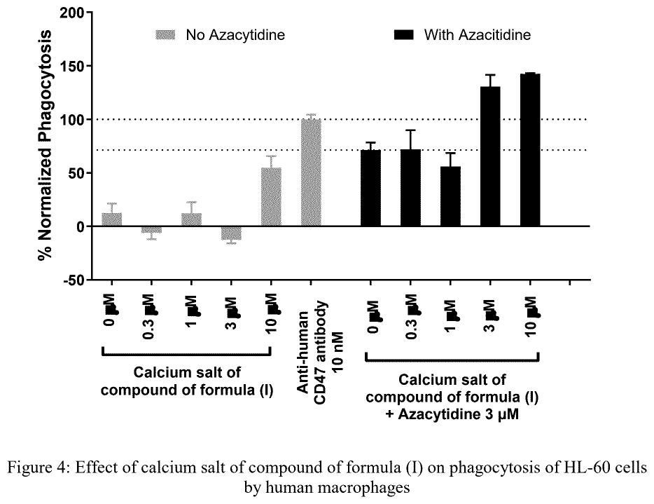

Effect of varying doses of Calcium salt of compound of formula (I) on phagocytic potential of human macrophages was tested in HL-60 cells treated with DMSO or 3 μM azacitidine. The results demonstrated that Calcium salt of compound of formula (I) treatment in combination with 3 μM azacitidine increased phagocytosis of HL-60 cells in dose dependent manner. In brief, Calcium salt of compound of formula (I) treatment alone increased phagocytosis of HL-60 cells by 54.79% at 10 μM concentrations. 3 μM azacitidine treatment of HL-60 cells increased phagocytosis by 71.26%. However, combination treatment of Calcium salt of compound of formula (I) and azacitidine (3 μM) further improved the phagocytosis of HL-60 cells (from 54.79% with 10 μM Calcium salt of compound of formula (I) alone to 142.53% with azacitidine combination) when compared to either treatment alone. Percent increase in normalized phagocytosis was plotted in a graph using GraphPad Prism and presented in the FIG. 4 and Table 6.

TABLE 6
Normalized phagocytosis of HL-60 cells with human macrophages

| | % Normalized phagocytosis | |
|---|---|---|
| Sample ID | Replicate 1 | Replicate 2 |
| Calcium salt of compound of formula (I) 10 μM | 62.35 | 47.13 |
| Calcium salt of compound of formula (I) 3 μM | −10.34 | −14.94 |
| Calcium salt of compound of formula (I) 1 μM | 19.54 | 4.98 |
| Calcium salt of compound of formula (I) 0.3 μM | −1.92 | −10.34 |
| Calcium salt of compound of formula (I) 10 μM + | 142.91 | 142.15 |
| Azacitidine 3 μM | | |
| Calcium salt of compound of formula (I) 3 μM + | 122.99 | 138.31 |
| Azacitidine 3 μM | | |
| Calcium salt of compound of formula (I) 1 μM + | 64.75 | 47.13 |
| Azacitidine 3 μM | | |
| Calcium salt of compound of formula (I) 0.3 μM + | 59.39 | 84.67 |
| Azacitidine 3 μM | | |
| No antibody | 18.77 | 6.51 |
| Anti-human CD47 antibody 10 nM | 103.07 | 96.93 |
| Isotype control 10 nM | 12.64 | −12.64 |
| Azacitidine 3 μM | 76.25 | 66.28 |

Conclusion: Treatment with Calcium salt of compound of formula (I) in combination with azacitidine resulted in synergistic increase in phagocytosis of HL-60 cells by human macrophages in dose dependent manner.

### EMBODIMENTS

The present disclosure provides for the following embodiments:

Embodiment 1. A CD47-SIRPα blocking agent, selected from the group consisting of the compound of formula (I):

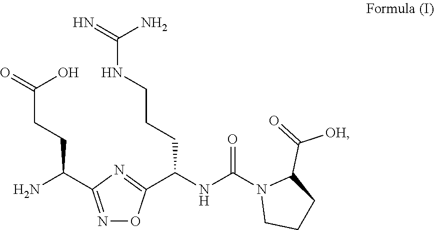

stereoisomers thereof, and pharmaceutically acceptable salts, solvates, amides and esters of the compound of formula (I) and stereoisomer thereof; for use in a method of treating or delaying progression of a disease or a disorder mediated by CD47-SIRPα pathway in a human subject, wherein the method comprises oral administration of the CD47-SIRPα blocking agent, and a therapeutically effective dose of Azacitidine, or a pharmaceutically acceptable salt thereof.

Embodiment 2. The CD47-SIRPα blocking agent for use according to Embodiment 1, wherein the salt of compound of formula (I) is selected from Calcium, Magnesium, Potassium and Sodium salts of the compound of formula (I), preferably a Calcium salt of compound of formula (I).

Embodiment 3. The CD47-SIRPα blocking agent for use according to Embodiment 1 or 2, wherein the disease or disorder mediated by CD47-SIRPα pathway is a cancer.

Embodiment 4. The CD47-SIRPα blocking agent for use according to any one of the preceding Embodiments 1-3, wherein the disease or disorder mediated by CD47-SIRPα pathway is a hematological cancer.

Embodiment 5. The CD47-SIRPα blocking agent for use according to any one of the preceding Embodiments 1-4, wherein the disease or disorder mediated by CD47-SIRPα pathway is leukemia.

Embodiment 6. The CD47-SIRPα blocking agent for use according to any one of the preceding Embodiment 1-5, wherein the disease or disorder mediated by CD47-SIRPα pathway is Acute Myeloid Leukemia (AML) or Myelodysplastic syndrome (MDS).

Embodiment 7. The CD47-SIRPα blocking agent for use according to Embodiment 6, wherein the subject is a newly diagnosed subject or a subject suffering from a relapsed and/or refractory AML.

Embodiment 8. The CD47-SIRPα blocking agent for use according to Embodiment 7, wherein the subject suffering from a relapsed and/or refractory AML has received at least one line of previous therapy and is eligible for one or more second or later line(s) of treatment.

Embodiment 9. The CD47-SIRPα blocking agent for use according to Embodiment 8, wherein the at least one line of previous therapy does not comprise a hypomethylating agent.

Embodiment 10. The CD47-SIRPα blocking agent for use according to any one of Embodiments 8-9, wherein the one of the second or later line of treatments is a therapeutically effective dose of Azacitidine, or a pharmaceutically acceptable salt thereof.

Embodiment 11. The CD47-SIRPα blocking agent for use according to Embodiment 7, wherein the newly diagnosed subject suffering from AML is not eligible for intensive chemotherapy.

Embodiment 12. The CD47-SIRPα blocking agent for use according to any one of the preceding Embodiment 1-11, wherein the treatment is characterized by the treated subject achieving at least one of the following response criteria according to European Leukemia Net (ELN-2017) after at least one treatment cycle comprising 28 days of treatment:
a. Complete remission;
b. Complete remission with incomplete hematologic recovery;
c. Morphologic leukemia free state;
d. Partial remission; or
e. Stable Disease lasting 3 months or more;

Embodiment 13. The CD47-SIRPα blocking agent for use according to any one of the preceding Embodiment 1-6, wherein the subject is an intermediate risk, high risk, or a very high-risk MDS subject who is eligible to receive Azacitidine.

Embodiments 14. The CD47-SIRPα blocking agent for use according to Embodiment 13, wherein the subject suffering from MDS has a Revised International Prognostic Scoring System (IPSS-R)≥3.5.

Embodiment 15. The CD47-SIRPα blocking agent for use according to any one of Embodiments 13 or 14, wherein the subject has not been previously treated with a hypomethylating agent.

Embodiment 16. The CD47-SIRPα blocking agent for use according to any one of the preceding Embodiment 13-15, wherein the subject suffering from MDS is eligible for one or more second or later line(s) of treatment.

Embodiment 17. The CD47-SIRPα blocking agent for use according to Embodiment 16, wherein one of the second or later line of treatments is a therapeutically effective dose of Azacitidine, or a pharmaceutically acceptable salt thereof.

Embodiment 18. The CD47-SIRPα blocking agent for use according to any one of the preceding Embodiments 1-6 or 13-17, wherein the disease or disorder mediated by CD47-SIRPα pathway is Myelodysplastic syndrome (MDS), and wherein the treatment is characterized by the treated subject achieving at least one of the following response criteria according to International Working Group (IWG) 2006:
i. Complete remission;
ii. Partial remission;
iii. Stable disease; or
iv. Hematological Improvement.

Embodiment 19. The CD47-SIRPα blocking agent for use according to any one of the preceding Embodiments 1-17, wherein the human subject has an Eastern Cooperative Oncology Group (ECOG) Performance Status Scale of 0-2.

Embodiment 20. The CD47-SIRPα blocking agent for use according to any one of the preceding Embodiments 1-19, wherein the human subject meets at least one of the criteria a) and b):
a) bone marrow assessment characterized by:
   i. WBC<20,000/μL, wherein hydroxyurea can be given to bring WBC count to <20,000/μl;
   ii. Platelet count≥50,000/μL without transfusion support; and/or
   iii. Hemoglobin≥9 g/dL, wherein transfusion is allowed to achieve this Hb; and/or,
b) organ function characterized by:
   i. Total Bilirubin≤1.5×Upper Limit Normal (ULN); or ≤2.5×ULN if the subject has Gilbert's syndrome;
   ii. AST (SGOT)≤3×ULN (≤5×ULN if the subject has liver metastases);
   iii. ALT (SGPT)≤3×ULN (≤5×ULN if the subject liver metastases);
   iv. Creatinine clearance (CrCl)≥60 mL/min; and/or
   v. Albumin≥3.0 g/dL.

Embodiment 21. The CD47-SIRPα blocking agent for use according to any one of the preceding Embodiments 1-20, wherein the CD47-SIRPα blocking agent and Azacitidine are administered simultaneously or sequentially.

Embodiment 22. The CD47-SIRPα blocking agent for use according to any one of the preceding Embodiments 1-21, wherein the CD47-SIRPα blocking agent is administered to the subject via an oral route.

Embodiment 23. The CD47-SIRPα blocking agent for use according to any one of the preceding Embodiments 1-22, wherein Azacitidine is administered to the subject via an oral route or a parenteral route, preferably via a subcutaneous injection or an intravenous infusion.

Embodiment 24. The CD47-SIRPα blocking agent for use according to any one of the preceding Embodiment 1-23, wherein the method comprises the administration of the compound of formula (I) at a total daily dose not exceeding 800 mg, preferably not exceeding 600 mg, more preferably not exceeding 500 mg, most preferably not exceeding 400 mg or the administration of a stereoisomer of the compound of formula (I) or a salt, solvate, amide or ester of the compound of formula (I) or stereoisomer thereof at the molar equivalent dose.

Embodiment 25. The CD47-SIRPα blocking agent for use according to any one of the preceding Embodiments 1-24, wherein the compound of formula (I) is administered to the subject once or twice daily.

Embodiment 26. The CD47-SIRPα blocking agent for use according to any one of the preceding Embodiments 1-25, wherein the method comprises the administration of the compound of formula (I) at a dose of 100 to 400 mg twice daily, preferably 100 mg, 200 mg, 300 mg, or 400 mg twice daily, or the twice daily administration of a stereoisomer of the compound of formula (I) or a salt, solvate, amide or ester of the compound of formula (I) or stereoisomer thereof at the molar equivalent dose.

Embodiment 27. The CD47-SIRPα blocking agent for use according to any one of the preceding Embodiment 1-26, wherein the method comprises the administration of the compound of formula (I) at a dose of 200 to 800 mg once daily, preferably 200 mg, 400 mg, 600 mg, or 800 mg once daily, or the once daily administration of a stereoisomer of the compound of formula (I) or a salt, solvate, amide or ester of the compound of formula (I) or stereoisomer thereof at the molar equivalent dose.

Embodiment 28. The CD47-SIRPα blocking agent for use according to any one of the preceding Embodiment 1-27, wherein the method further comprises the step of modifying the treatment in case the subject shows sign of prohibitive toxicity and/or disease progression.

Embodiment 29. The CD47-SIRPα blocking agent for use according to Embodiment 28, wherein the step of modifying the treatment comprises:
i. a reduction in the total daily dose and/or dosing frequency, wherein the treatment is continued at a relatively lower daily dose and/or a reduced dosing frequency of the compound of formula (I);
ii. a dose hold, wherein the treatment is interrupted until the subject displays an improvement in prohibitive toxicity, and then reinitiating the treatment with the same daily dose and/or dosing frequency of the compound of formula (I); or
iii. a schedule change, wherein the treatment is interrupted until the subject displays improvement in prohibitive toxicity, and then reinitiating the treatment with a relatively lower daily dose and/or a reduced dosing frequency of the compound of formula (I).

Embodiment 30. The CD47-SIRPα blocking agent for use according to Embodiment 28 or 29, wherein the prohibitive toxicity is a dose limiting toxicity (DLT).

Embodiment 31. The CD47-SIRPα blocking agent for use according to any one of Embodiments 28-30, wherein the dose limiting toxicity (DLT) is characterized by one or more of the toxicities selected from:
a. Positive for Hy's law (AST or ALT≥3×ULN with concomitant Total Bilirubin≥2×ULN, without findings of cholestasis on radiological scans and no other reasons for increase in AST/ALT and bilirubin);
b. Grade 4 neutropenia (ANC<500/μL) in the absence of persistent disease (AML/MDS); and,
c. Any other Grade 3 or higher clinically significant toxicity (except alopecia or nail changes), as graded by National Cancer Institute Common Terminology Criteria for Adverse Events (NCI CTCAE) v 5.0 which is considered as “not clearly or incontrovertibly related to the underlying malignancy, any other co-morbidities or concomitant medications or extraneous causes”.

Embodiment 32. The CD47-SIRPα blocking agent for use according to Embodiment 29, wherein the lower daily dose is a dose selected from three-fourth, half and one-third of the dose at which the prohibitive toxicity is displayed.

Embodiment 33. The CD47-SIRPα blocking agent for use according to Embodiment 29, wherein the reduced dosing frequency is half, one-third, or one-fourth of the dosing frequency at which the prohibitive toxicity is displayed.

Embodiment 34. The CD47-SIRPα blocking agent for use according to Embodiment 29, wherein the step of modifying the treatment, by a reduction in the total daily dose and/or dosing frequency, is done up to 3 times before the treatment is terminated.

Embodiment 35. The CD47-SIRPα blocking agent for use according to Embodiment 28, wherein the step of modifying the treatment comprises one or more escalations of total daily dose and/or dosing frequency of the CD47-SIRPα blocking agent, until the subject displays a prohibitive toxicity.

Embodiment 36. The CD47-SIRPα blocking agent for use according to Embodiment 35, wherein the escalated total daily dose is up to 1.5 times, up to 2-times, up to 3-times, or up to 4-times of the total daily dose at which the disease progression was displayed.

Embodiment 37. The CD47-SIRPα blocking agent for use according to Embodiment 35, wherein the escalated dosing frequency is up to 2-times, 3-times or 4-times of the dosing frequency at which the disease progression is displayed.

Embodiment 38. The CD47-SIRPα blocking agent for use according to any one of Embodiments 35-37, wherein the escalation of the total daily dose and/or dosing frequency is done up to 3 times before the treatment is terminated.

Embodiment 39. The CD47-SIRPα blocking agent for use according to Embodiment 28, wherein the disease progression for a subject suffering from AML is characterized by the treated subject failing to maintain at least one of the following response criteria, or downgrading to a lower response criteria according to European Leukemia Net (ELN-2017) is:
a. Complete remission;
b. Complete remission with incomplete hematologic recovery;
c. Morphologic leukemia free state;
d. Partial remission; or
e. Stable Disease lasting 3 months or more.

Embodiment 40. The CD47-SIRPα blocking agent for use according to Embodiment 28, wherein the disease progression for a subject suffering from MDS is characterized by the treated subject failing to maintain at least one of the following response criteria, or downgrading to a lower response criteria according to International Working Group (IWG) 2006:
i. Complete remission;
ii. Partial remission;
iii. Stable disease; or
iv. Hematological Improvement.

Embodiment 41. The CD47-SIRPα blocking agent for use according to any one of the preceding Embodiments 1-40, wherein a therapeutically effective dose of Azacitidine is administered as subcutaneous injection or intravenous infusion, daily for 7 days in a 28 days of treatment cycle for a minimum of 4 to 6 treatment cycles.

Embodiment 42. The CD47-SIRPα blocking agent for use according to any one of the preceding Embodiments 1-41, wherein a therapeutically effective dose of Azacitidine is administered orally once daily, for 14 days in a 28 days of treatment cycle for a minimum of 3 to 4 treatment cycles.

Embodiment 43. The CD47-SIRPα blocking agent for use according to any one of the preceding claims 1-42, wherein Azacitidine is subcutaneously (SC) or intravenously (IV) administered on days 1-5 followed by 2 days off, and then two days again in a 28 days of treatment cycle.

Embodiment 44. The CD47-SIRPα blocking agent for use according to any one of the preceding Embodiments 1-43, wherein the method comprises the administration of the compound of formula (I) at a dose of 200 mg to 400 mg, preferably 200 mg, 300 mg or 400 mg twice daily for at least 28 days, or the twice daily administration, for at least 28 days, of a stereoisomer of the compound of formula (I) or a salt, solvate, amide or ester of the compound of formula (I) or stereoisomer thereof at the molar equivalent dose.

Embodiment 45. The CD47-SIRPα blocking agent for use according to any one of the preceding Embodiments 1-44, wherein the method comprises the administration of the calcium salt of compound of formula (I) at a dose of 300 mg twice daily at the molar equivalent dose.

Embodiment 46. A method of treating or delaying progression of a disease or a disorder mediated by CD47-SIRPα pathway in a human subject, wherein the method comprises the administration of a CD47-SIRPα blocking agent selected from the group consisting of the compound of formula (I):

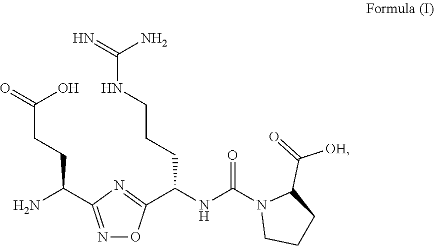

stereoisomers thereof, and a molar equivalent amount of pharmaceutically acceptable salts, solvates, amides and esters of the compound of formula (I), and stereoisomers thereof; as well as the administration of Azacitidine, or a pharmaceutically acceptable salt thereof.

Embodiment 47. The method of treatment or delaying progression according to Embodiment 46, wherein the salt of compound of formula (I) is selected from the group consisting of Calcium, Magnesium, Potassium and Sodium salts of the compound of formula (I), preferably a Calcium salt of compound of formula (I).

Embodiment 48. The method of treatment or delaying progression according to Embodiment 46 or 47, wherein the disease or disorder mediated by CD47-SIRPα pathway is a cancer.

Embodiment 49. The method of treatment or delaying progression according to any one of the preceding Embodiments 46-48, wherein the disease or disorder mediated by CD47-SIRPα pathway is a hematological cancer.

Embodiment 50. The method of treatment or delaying progression according to any one of the preceding Embodiments 46-49, wherein the disease or disorder mediated by CD47-SIRPα pathway is leukemia.

Embodiment 51. The method of treatment or delaying progression according to any one of the preceding Embodiments 46-50, wherein the disease or disorder mediated by CD47-SIRPα pathway is Acute Myeloid Leukemia (AML) or Myelodysplastic syndrome (MDS).

Embodiment 52. The method of treatment or delaying progression according to Embodiment 51, wherein the subject is a newly diagnosed subject or a subject suffering from a relapsed and/or refractory AML.

Embodiment 53. The method of treatment or delaying progression according to Embodiment 52, wherein the subject suffering from a relapsed and/or refractory AML has received at least one line of previous therapy and is eligible for one or more second or later line(s) of treatment.

Embodiment 54. The method of treatment or delaying progression according to Embodiment 53, wherein the at least one line of previous therapy does not comprise a hypomethylating agent.

Embodiment 55. The method of treatment or delaying progression according to any one of Embodiments 52-53, wherein the one of the second or later line of treatments is a therapeutically effective dose of Azacitidine, or a pharmaceutically acceptable salt thereof.

Embodiment 56. The method of treatment or delaying progression according to Embodiment 52, wherein the newly diagnosed subject suffering from AML is not eligible for intensive chemotherapy.

Embodiment 57. The method of treatment or delaying progression according to any one of the preceding Embodiments 46-56, wherein the treatment is characterized by the treated subject achieving at least one of the following response criteria according to European Leukemia Net (ELN-2017):
a. Complete remission;
b. Complete remission with incomplete hematologic recovery;
c. Morphologic leukemia free state;
d. Partial remission; or
e. Stable Disease lasting 3 months or more;

Embodiment 58. The method of treatment or delaying progression according to any one of the preceding Embodiments 46-51, wherein the subject is an intermediate risk, high risk, or a very high-risk MDS subject who is eligible to receive Azacitidine.

Embodiment 59. The method of treatment or delaying progression according to Embodiment 58, wherein the subject suffering from MDS has a Revised International Prognostic Scoring System (IPSS-R)≥3.5.

Embodiment 60. The method of treatment or delaying progression according to any one of Embodiments 58 or 59, wherein the subject has not been previously treated with a hypomethylating agent.

Embodiment 61. The method of treatment or delaying progression according to any one of the preceding Embodiments 58-60, wherein the subject suffering from MDS is eligible for one or more second or later line(s) of treatment.

Embodiment 62. The method of treatment or delaying progression according to Embodiment 61, wherein the one of the second or later line of treatments is a therapeutically effective dose of Azacitidine, or a pharmaceutically acceptable salt thereof.

Embodiment 63. The method of treatment or delaying progression according to any one of the preceding Embodiments 46-51 or 58-62, wherein the disease or disorder mediated by CD47-SIRPα pathway is Myelodysplastic syndrome (MDS), and wherein the treatment is characterized by the treated subject achieving at least one of the following response criteria according to International Working Group (IWG) 2006:
i. Complete remission;
ii. Partial remission;
iii. Stable disease; or
iv. Hematological Improvement.

Embodiment 64. The method of treatment or delaying progression according to any one of the preceding Embodiments 46-62, wherein the human subject has a Eastern Cooperative Oncology Group (ECOG) Performance Status Scale of 0-2.

Embodiment 65. The method of treatment or delaying progression according to any one of the preceding Embodiments 46-64, wherein the human subject meets at least one of the criteria a) and b):
a) bone marrow assessment characterized by:
   i. WBC<20,000/μL, wherein hydroxyurea can be given to bring WBC count to <20,000/μl;
   ii. Platelet count≥50,000/μL without transfusion support; and/or
   iii. Hemoglobin≥9 g/dL, wherein transfusion is allowed to achieve this Hb; and/or,
b) organ function characterized by:
   i. Total Bilirubin≤1.5×Upper Limit Normal (ULN); or ≤2.5×ULN if the subject has Gilbert's syndrome;
   ii. AST (SGOT)≤3×ULN (≤5×ULN if the subject has liver metastases);
   iii. ALT (SGPT)≤3×ULN (≤5×ULN if the subject liver metastases);
   iv. Creatinine clearance (CrCl)≥60 mL/min; and/or
   v. Albumin≥3.0 g/dL.

Embodiment 66. The method of treatment or delaying progression according to any one of the preceding Embodiments 46-65, wherein the CD47-SIRPα blocking agent and Azacitidine are administered simultaneously or sequentially.

Embodiment 67. The method of treatment or delaying progression according to any one of the preceding Embodiments 46-66, wherein the CD47-SIRPα blocking agent is administered to the subject via an oral route.

Embodiment 68. The method of treatment or delaying progression according to any one of the preceding Embodiments 46-67, wherein Azacitidine is administered to the subject via an oral or a parenteral route, preferably via a subcutaneous injection or an intravenous infusion.

Embodiment 69. The method of treatment or delaying progression according to any one of the preceding Embodiments 46-68, wherein the method comprises the administration of the compound of formula (I) at a total daily dose not exceeding 800 mg, preferably not exceeding 600 mg, more preferably not exceeding 500 mg, most preferably not exceeding 400 mg, or the administration of a stereoisomer of the compound of formula (I) or a salt, solvate, amide or ester of the compound of formula (I) or stereoisomer thereof at the molar equivalent dose.

Embodiment 70. The method of treatment or delaying progression according to any one of the preceding Embodiments 46-69, wherein the compound of formula (I) is administered to the subject once daily or twice daily.

Embodiment 71. The method of treatment or delaying progression according to any one of the preceding Embodiments 46-70, wherein the method comprises the administration of the compound of formula (I) at a dose of 100 to 400 mg twice daily, preferably 100 mg, 200 mg, 300 mg, or 400 mg twice daily, or the twice daily administration of a stereoisomer of the compound of formula (I) or a salt, solvate, amide or ester of the compound of formula (I) or stereoisomer thereof at the molar equivalent dose.

Embodiment 72. The method of treatment or delaying progression according to any one of the preceding Embodiments 46-71, wherein the method comprises the administration of the compound of formula (I) at a dose of 200 to 800 mg once daily, preferably 200 mg, 400 mg, 600 mg, or 800 mg once daily, or the once daily administration of a stereoisomer of the compound of formula (I) or a salt, solvate, amide or ester of the compound of formula (I) or stereoisomer thereof at the molar equivalent dose.

Embodiment 73. The method of treatment or delaying progression according to any one of the preceding Embodiments 46-72, wherein the method further comprises the step of modifying the treatment in case the subject shows signs of a prohibitive toxicity and/or disease progression.

Embodiment 74. The method of treatment or delaying progression according to Embodiment 73, wherein the step of modifying the treatment comprises:
i. a reduction in the total daily dose and/or dosing frequency, wherein the treatment is continued at a relatively lower daily dose and/or a reduced dosing frequency of the compound of formula (I);
ii. a dose hold, wherein the treatment is interrupted until the subject displays an improvement in prohibitive toxicity, and then reinitiating the treatment with the same daily dose and/or dosing frequency of the compound of formula (I); or
iii. a schedule change, wherein the treatment is interrupted until the subject displays improvement in prohibitive toxicity, and then reinitiating the treatment with a relatively lower daily dose and/or a reduced dosing frequency of the compound of formula (I).

Embodiment 75. The method of treatment or delaying progression according to Embodiment 73 or 74, wherein the prohibitive toxicity is a dose limiting toxicity (DLT).

Embodiment 76. The method of treatment or delaying progression according to any one of Embodiments 73-75, wherein the dose limiting toxicity (DLT) is characterized by one or more of the toxicities selected from:
i. Positive for Hy's law (AST or ALT≥3×ULN with concomitant Total Bilirubin≥2×ULN, without findings of cholestasis on radiological scans and no other reasons for increase in AST/ALT and bilirubin);
ii. Grade 4 neutropenia (ANC<500/μL) in the absence of persistent disease (AML/MDS); and
iii. Any other Grade 3 or higher clinically significant toxicity (except alopecia or nail changes), as graded by National Cancer Institute Common Terminology Criteria for Adverse Events (NCI CTCAE) v 5.0 which is considered as “not clearly or incontrovertibly related to the underlying malignancy, any other co-morbidities or concomitant medications or extraneous causes”.

Embodiment 77. The method of treatment or delaying progression according to Embodiment 76, wherein the lower daily dose is a dose selected from three-fourth, half and one-third of the dose at which the prohibitive toxicity is displayed.

Embodiment 78. The method of treatment or delaying progression use according to Embodiment 76, wherein the reduced dosing frequency is half, one-third, or one-fourth of the dosing frequency at which the prohibitive toxicity is displayed.

Embodiment 79. The method of treatment or delaying progression according to any one of Embodiments 76-78, wherein the step of modifying the treatment, by a reduction in the total daily dose and/or dosing frequency, is done up to 3 times before the treatment is terminated.

Embodiment 80. The method of treatment or delaying progression according to Embodiment 73, wherein the step of modifying the treatment comprises one or more escalations of total daily dose and/or dosing frequency of the CD47-SIRPα blocking agent until the subject displays a prohibitive toxicity.

Embodiment 81. The method of treatment or delaying progression according to Embodiment 80, wherein the escalated total daily dose is up to one and half times, up to two-times, up to three-times, or up to four-times of the total daily dose at which the disease progression is displayed.

Embodiment 82. The method of treatment or delaying progression use according to Embodiment 80, wherein the escalated dosing frequency is up to two-times, three-times or four-times of the dosing frequency at which the disease progression is displayed.

Embodiment 83. The method of treatment or delaying progression according to any one of Embodiments 80-82, wherein the escalation of the total daily dose and/or dosing frequency is done up to three times before the treatment is terminated.

Embodiment 84. The CD47-SIRPα blocking agent for use according to any one of Embodiments 73 or 80-83, wherein the disease progression for a subject suffering from AML is characterized by treated subject failing to maintain at least one of the following response criteria, or downgrading to a lower response criteria according to European Leukemia Net (ELN-2017):
a. Complete remission;
b. Complete remission with incomplete hematologic recovery;
c. Morphologic leukemia free state;
d. Partial remission; or
e. Stable Disease lasting 3 months or more.

Embodiment 85. The CD47-SIRPα blocking agent for use according to any one of Embodiments 73 or 80-83, wherein the disease progression for a subject suffering from MDS is characterized by treated subject failing to maintain at least one of the following response criteria, or downgrading to a lower response criteria according to International Working Group (IWG) 2006:
i. Complete remission;
ii. Partial remission;
iii. Stable disease; or
iv. Hematological Improvement.

Embodiment 86. The method of treatment or delaying progression according to any one of the preceding Embodiments 46-85, wherein a therapeutically effective dose of Azacitidine is administered as subcutaneous injection or intravenous infusion, daily for 7 days in a 28 days treatment cycle for a minimum of 4 to 6 treatment cycles.

Embodiment 87. The method of treatment or delaying progression according to any one of the preceding Embodiments 46-85, wherein a therapeutically effective dose of Azacitidine is administered orally once daily, for 14 days in a 28 days treatment cycle for a minimum of 3 to 4 treatment cycles.

Embodiment 88. The method of treatment or delaying progression according to any one of the preceding Embodiments 46-85, wherein Azacitidine is subcutaneously (SC) or intravenously (IV) administered on days 1-5 followed by 2 days off, and then two days again in a 28 days of treatment cycle.

Embodiment 89. The method of treatment or delaying progression according to any one of the preceding Embodiments 46-88, wherein the method comprises the administration of the calcium salt of compound of formula (I) at a dose of 300 mg twice daily at the molar equivalent dose.

Embodiment 90. A kit comprising:
(i) a plurality of pharmaceutical compositions, preferably in unit dosage form, comprising a CD47-SIRPα blocking agent, selected from the group consisting of the compound of formula (I):

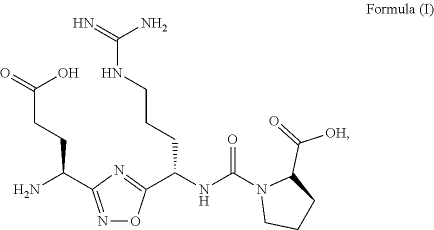

stereoisomers thereof, and pharmaceutically acceptable salts, solvates, amides and esters of the compound of formula (I) and stereoisomer thereof;
(ii) optionally, a pharmaceutical composition of Azacitidine, or a pharmaceutically acceptable salt thereof; and,
(iii) a package and/or package insert, said package and/or insert having instructions printed thereon to use said pharmaceutical compositions in a method of treating or delaying progression of a disease or a disorder mediated by CD47-SIRPα in a subject.

Embodiment 91. The kit according to Embodiment 90, wherein the salt of CD47-SIRPα blocking agent is a salt selected from the group consisting of Calcium, Magnesium, Potassium and Sodium salts of the compound of formula (I), preferably a Calcium salt of compound of formula (I).

Embodiment 92. The kit according to Embodiment 90 or 91, wherein the instructions provide that the disease or disorder mediated by CD47-SIRPα pathway is a cancer.

Embodiment 93. The kit according to any one of the preceding Embodiments 90-92, wherein the instructions provide that the disease or disorder mediated by CD47-SIRPα pathway is a hematological cancer.

Embodiment 94. The kit according to any one of the preceding Embodiments 90-93, wherein the instructions provide that the disease or disorder mediated by CD47-SIRPα pathway is leukemia.

Embodiment 95. The kit according to any one of the preceding Embodiments 90-94, wherein the instructions provide that the disease or disorder mediated by CD47-SIRPα pathway is Acute Myeloid Leukemia (AML) or Myelodysplastic syndrome (MDS).

Embodiment 96. The kit according to Embodiment 95, wherein the instructions provide that the AML is a newly diagnosed AML or a relapsed and/or refractory AML.

Embodiment 97. The kit according to Embodiment 95, wherein the instructions provide that the subject suffering from the relapsed and/or refractory AML has received at least one line of previous therapy and is eligible for one or more second or later line(s) of treatment.

Embodiment 98. The kit according to Embodiment 97, wherein the instructions provide that the at least one line of previous therapy does not comprise a hypomethylating agent.

Embodiment 99. The kit according to any one of the preceding Embodiments 97-98, wherein the instructions provide that the one of the second or later line of treatments is a therapeutically effective dose of Azacitidine, or a pharmaceutically acceptable salt thereof.

Embodiment 100. The kit according to Embodiment 96, wherein the instructions provide that the subject suffering from the said newly diagnosed AML is not eligible for intensive chemotherapy.

Embodiment 101. The kit according to any one of the preceding Embodiments 96-100, wherein the instructions further provide that the delay of progression or improvement in the signs and symptoms of the disorder is assessed, based on at least one of the following response criteria according to European Leukemia Net (ELN-2017):
a. Complete remission;
b. Complete remission with incomplete hematologic recovery;
c. Morphologic leukemia free state;
d. Partial remission; or
e. Stable Disease lasting 3 months or more;

Embodiment 102. The kit according to Embodiment 95, wherein the instructions provide that the disease or disorder mediated by CD47-SIRPα pathway is an intermediate risk, high risk, or a very high-risk Myelodysplastic syndrome (MDS), and wherein the subject suffering from the said MDS is eligible to receive Azacitidine.

Embodiment 103. The kit according to Embodiment 102, wherein the instructions provides that the subject has a Revised International Prognostic Scoring System (IPSS-R)≥3.5.

Embodiment 104. The kit according to any one of Embodiments 102 or 103, wherein the instructions provides that the subject has not been previously treated with a hypomethylating agent.

Embodiment 105. The kit according to any one of the preceding Embodiments 102-104, wherein the instructions provide that the subject is eligible for one or more second or later line(s) of treatment.

Embodiment 106. The kit according to Embodiment 105, wherein the instructions provide that the one of the second or later line of treatments is a therapeutically effective dose of Azacitidine, or a pharmaceutically acceptable salt thereof.

Embodiment 107. The kit according to Embodiment 106, wherein the instructions further provide that the delay of progression or improvement in the signs and symptoms of the disorder is assessed, based on at least one of the following response criteria according to International Working Group (IWG) 2006:
a. Complete remission;
b. Partial remission;
c. Stable disease; or
d. Hematological Improvement.

Embodiment 108. The kit according to any one of the preceding Embodiments 90-107, wherein the instructions further provide that the subject has a Eastern Cooperative Oncology Group (ECOG) Performance Status Scale of 0-2.

Embodiment 109. The kit according to any one of the preceding Embodiments 90-108, wherein the instructions further provide that the subject meets at least one of the criteria a) and b):
a) bone marrow assessment characterized by:
   i. WBC<20,000/μL, wherein hydroxyurea can be given to bring WBC count to <20,000/μl;
   ii. Platelet count≥50,000/μL without transfusion support; and
   iii. Hemoglobin≥9 g/dL, wherein transfusion is allowed to achieve this Hb; and/or,
b) organ function characterized by:
   i. Total Bilirubin≤1.5×Upper Limit Normal (ULN); or ≤2.5×ULN if the subject has Gilbert's syndrome;
   ii. AST (SGOT)≤3×ULN (≤5×ULN if the subject has liver metastases);
   iii. ALT (SGPT)≤3×ULN (≤5×ULN if the subject liver metastases);
   iv. Creatinine clearance (CrCl)≥60 mL/min; and
   v. Albumin≥3.0 g/dL.

Embodiment 110. The kit according to any one of the preceding Embodiments 90-109, wherein the instructions further provide that the CD47-SIRPα blocking agent and Azacitidine are administered simultaneously or sequentially.

Embodiment 111. The kit according to any one of the preceding Embodiments 90-110, wherein the instructions provide that the CD47-SIRPα blocking agent is administered to the subject via an oral route.

Embodiment 112. The kit according to any one of the preceding Embodiments 90-111, wherein the instructions provide that the Azacitidine is administered to the subject via an oral or a parenteral route, preferably via a subcutaneous injection or an intravenous infusion.

Embodiment 113. The kit according to any one of the preceding Embodiments 90-112, wherein the instructions further provide that the compound of formula (I) is administered at a total daily dose not exceeding 800 mg, preferably not exceeding 600 mg, more preferably not exceeding 500 mg, most preferably not exceeding 400 mg, or that a stereoisomer of the compound of formula (I) or a salt, solvate, amide or ester of the compound of formula (I) or stereoisomer thereof is administered at the molar equivalent dose.

Embodiment 114. The kit according to any one of the preceding Embodiments 90-113, wherein the instructions further provide that compound of formula (I) is administered to the subject once daily or twice daily.

Embodiment 115. The kit according to any one of the preceding Embodiments 90-114, wherein the one instructions further provide that the compound of formula (I) is administered at a dose of 100 to 400 mg twice daily, preferably 100 mg, 200 mg, 300 mg, or 400 mg twice daily, or that a stereoisomer of the compound of formula (I) or a salt, solvate, amide or ester of the compound of formula (I) or stereoisomer thereof is administered twice daily at the molar equivalent dose.

Embodiment 116. The kit according to any one of the preceding Embodiments 90-115, wherein the instructions further provide that compound of formula (I) is administered at a dose of 200 to 800 mg once daily, preferably 200 mg, 400 mg, 600 mg, or 800 mg once daily, or that a stereoisomer of the compound of formula (I) or a salt, solvate, amide or ester of the compound of formula (I) or stereoisomer thereof is administered once daily at the molar equivalent dose.

Embodiment 117. The kit according to any one of the preceding Embodiments 90-116, wherein the instructions provide that the use further comprises a step of modifying the treatment in case of a prohibitive toxicity and/or disease progression.

Embodiment 118. The kit according to Embodiment 117, wherein the instructions provide that the step of modifying the treatment comprises:
a. a reduction in the total daily dose and/or dosing frequency of the compound of formula (I);
b. a dose hold until the subject displays an improvement in prohibitive toxicity, and then continuing with the same daily dose and/or dosing frequency of the compound of formula (I); or
c. a schedule change comprising a dose hold until the subject displays improvement in prohibitive toxicity, and then continuing with a relatively lower daily dose and/or a reduced dosing frequency of the compound of formula (I).

Embodiment 119. The kit according to Embodiment 117 or 118, wherein the instructions further provide that the prohibitive toxicity is a dose limiting toxicity (DLT).

Embodiment 120. The kit according to any one of Embodiments 117-119, wherein the instructions further provide that prohibitive toxicity is a dose limiting toxicity (DLT) characterized by one or more of the toxicities selected from:
a. Positive for Hy's law (AST or ALT≥3×ULN with concomitant Total Bilirubin≥2×ULN, without findings of cholestasis on radiological scans and no other reasons for increase in AST/ALT and bilirubin);
b. Grade 4 neutropenia (ANC<500/μL) in the absence of persistent disease (AML/MDS); and
c. Any other Grade 3 or higher clinically significant toxicity (except alopecia or nail changes), as graded by National Cancer Institute Common Terminology Criteria for Adverse Events (NCI CTCAE) v 5.0 which is considered as “not clearly or incontrovertibly related to the underlying malignancy, any other co-morbidities or concomitant medications or extraneous causes”.

Embodiment 121. The kit according to Embodiment 118, wherein the instructions provide that the lower daily dose is a dose selected from three-fourth, half and one-third of the dose at which the prohibitive toxicity is displayed.

Embodiment 122. The kit according to Embodiment 118, wherein the instructions provide that the reduced dosing frequency is half, one-third, or one-fourth of the dosing frequency at which the prohibitive toxicity is displayed.

Embodiment 123. The kit according to any one of Embodiments 118-120, wherein the instructions further provide that the step of modifying the treatment, by a reduction in the total daily dose and/or dosing frequency, is done up to three times before terminating the said use.

Embodiment 124. The kit according to Embodiment 117, wherein the instructions provide that that the step of modifying the treatment comprises one or more escalations of total daily dose and/or dosing frequency of the CD47-SIRPα blocking agent until the subject displays a prohibitive toxicity.

Embodiment 125. The kit according to Embodiment 124, wherein the instructions provide that the escalated total daily dose is up to one-and-a-half times, up to two-times, up to three-times, or up to four-times of the total daily dose at which the disease progression is displayed.

Embodiment 126. The kit according to Embodiment 124, wherein the instructions provide that the escalated dosing frequency is up to two-times, three-times or four-times of the dosing frequency at which the disease progression is displayed.

Embodiment 127. The kit according to any one of Embodiments 124-126, wherein the instructions provide that the escalation in the total daily dose and/or dosing frequency is done up to three times before the said use is terminated.

Embodiment 128. The kit according to any one of Embodiments 117 or 124-127, wherein the instructions provide that in a subject suffering from AML, the disease progression is characterized by the subject failing to maintain at least one of the following response criteria, or downgrading to a lower response criteria according to European Leukemia Net (ELN-2017):
a. Complete remission;
b. Complete remission with incomplete hematologic recovery;
c. Morphologic leukemia free state;
d. Partial remission; or
e. Stable Disease lasting 3 months or more.

Embodiment 129. The kit according to any one of Embodiments 117 or 124-127, wherein the instructions provide that in a subject suffering from MDS, the disease progression is characterized by the subject failing to maintain at least one of the following response criteria, or downgrading to a lower response criteria according to International Working Group (IWG) 2006:
a. Complete remission;
b. Partial remission;
c. Stable disease; or
d. Hematological Improvement.

Embodiment 130. The kit according to any one of the preceding Embodiments 90-129, wherein the instructions provide that a therapeutically effective dose of Azacitidine is administered as subcutaneous injection or intravenous infusion, daily for 7 days in a 28 days treatment cycle for a minimum of 4 to 6 treatment cycles.

Embodiment 131. The kit according to any one of the preceding Embodiments 90-129, wherein the instructions provide that a therapeutically effective dose of Azacitidine is administered orally once daily, for 14 days in a 28 days treatment cycle for a minimum of 3 to 4 treatment cycles.

Embodiment 132. The kit according to any one of the preceding Embodiments 90-129, wherein the instructions provide that Azacitidine is subcutaneously (SC) or intravenously (IV) administered on days 1-5 followed by 2 days off, and then two days again in a 28 days of cycle.

Embodiment 133. Use of a CD47-SIRPα blocking agent, selected from the group consisting of the compound of formula (I):

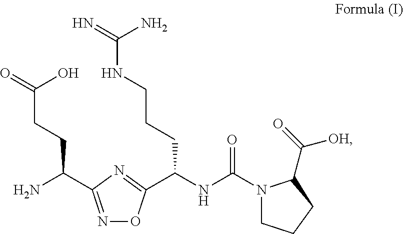

stereoisomers thereof, and pharmaceutically acceptable salts, solvates, amides and esters of the compound of formula (I) and stereoisomer thereof; in the manufacture of a pharmaceutical composition or medicament for use in a method of treating or delaying progression of a disease or a disorder mediated by CD47-SIRPα pathway in a human subject, wherein the method comprises the administration of the CD47-SIRPα blocking agent, as well as the administration of Azacitidine, or a pharmaceutically acceptable salt thereof.

Embodiment 134. A pharmaceutical composition comprising CD47-SIRPα blocking agent, selected from the group consisting of the compound of formula (I):

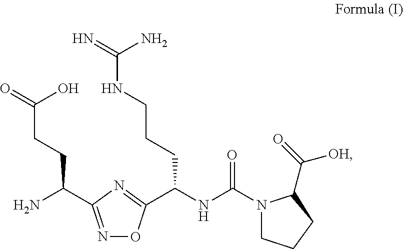

stereoisomers thereof, and pharmaceutically acceptable salts, solvates, amides and esters of the compound of formula (I) and stereoisomer thereof, for use in combination with another pharmaceutical composition comprising Azacitidine, in a method of treating or delaying the progression of Acute Myeloid Leukemia (AML) or Myelodysplastic Syndrome (MDS) in a human subject, wherein the method comprises administering to the subject in need thereof a compound of formula (I) at a total daily dose not exceeding 800 mg, preferably not exceeding 600 mg, more preferably not exceeding 500 mg, most preferably not exceeding 400 mg or the administration of a stereoisomer of the compound of formula (I) or a salt, solvate, amide or ester of the compound of formula (I) or stereoisomer thereof at the molar equivalent dose.

1. A CD47-SIRPα blocking agent, selected from the group consisting of the compound of formula (I): 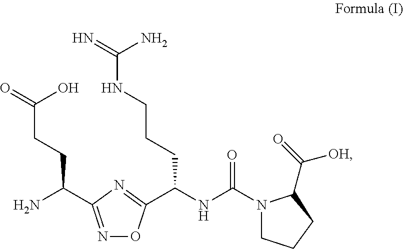
stereoisomers thereof, and pharmaceutically acceptable salts, solvates, amides and esters of the compound of formula (I) and stereoisomer thereof; for use in a method of treating or delaying progression of a disease or a disorder mediated by CD47-SIRPα pathway in a human subject, wherein the method comprises oral administration of the CD47-SIRPα blocking agent, and a therapeutically effective dose of Azacitidine, or a pharmaceutically acceptable salt thereof.

2. The CD47-SIRPα blocking agent for use according to claim 1, wherein the salt of compound of formula (I) is selected from Calcium, Magnesium, Potassium and Sodium salts of the compound of formula (I), preferably a Calcium salt of compound of formula (I).

3. The CD47-SIRPα blocking agent for use according to claim 1, wherein the disease or disorder mediated by CD47-SIRPα pathway is a cancer.

4. The CD47-SIRPα blocking agent for use according to claim 1, wherein the disease or disorder mediated by CD47-SIRPα pathway is a hematological cancer.

5. The CD47-SIRPα blocking agent for use according to claim 1, wherein the disease or disorder mediated by CD47-SIRPα pathway is leukemia.

6. The CD47-SIRPα blocking agent for use according to claim 1, wherein the disease or disorder mediated by CD47-SIRPα pathway is Acute Myeloid Leukemia (AML) or Myelodysplastic syndrome (MDS).

7. The CD47-SIRPα blocking agent for use according to claim 6, wherein the subject is a newly diagnosed subject or a subject suffering from a relapsed and/or refractory AML.

8. The CD47-SIRPα blocking agent for use according to claim 7, wherein the subject suffering from a relapsed and/or refractory AML has received at least one line of previous therapy and is eligible for one or more second or later line(s) of treatment.

9. The CD47-SIRPα blocking agent for use according to claim 8, wherein the at least one line of previous therapy does not comprise a hypomethylating agent.

10. The CD47-SIRPα blocking agent for use according to claim 8, wherein the one of the second or later line of treatments is a therapeutically effective dose of Azacitidine, or a pharmaceutically acceptable salt thereof.

11. The CD47-SIRPα blocking agent for use according to claim 7, wherein the newly diagnosed subject suffering from AML is not eligible for intensive chemotherapy.

12. The CD47-SIRPα blocking agent for use according to claim 1, wherein the treatment is characterized by the treated subject achieving at least one of the following response criteria according to European Leukemia Net (ELN-2017) after at least one treatment cycle comprising 28 days of treatment:
a. Complete remission;
b. Complete remission with incomplete hematologic recovery;
c. Morphologic leukemia free state;
d. Partial remission; or
e. Stable Disease lasting 3 months or more;

13. The CD47-SIRPα blocking agent for use according to claim 1, wherein the subject is an intermediate risk, high risk, or a very high-risk MDS subject who is eligible to receive Azacitidine.

14. The CD47-SIRPα blocking agent for use according to claim 13, wherein the subject suffering from MDS has a Revised International Prognostic Scoring System (IPSS-R)≥3.5.

15. The CD47-SIRPα blocking agent for use according to claim 13, wherein the subject has not been previously treated with a hypomethylating agent.

16. The CD47-SIRPα blocking agent for use according to claim 13, wherein the subject suffering from MDS is eligible for one or more second or later line(s) of treatment.

17. The CD47-SIRPα blocking agent for use according to claim 16, wherein one of the second or later line of treatments is a therapeutically effective dose of Azacitidine, or a pharmaceutically acceptable salt thereof.

18. The CD47-SIRPα blocking agent for use according to claim 1, wherein the disease or disorder mediated by CD47-SIRPα pathway is Myelodysplastic syndrome (MDS), and wherein the treatment is characterized by the treated subject achieving at least one of the following response criteria according to International Working Group (IWG) 2006:
i. Complete remission;
ii. Partial remission;
iii. Stable disease; or
iv. Hematological Improvement.

19. The CD47-SIRPα blocking agent for use according to claim 1, wherein the human subject has an Eastern Cooperative Oncology Group (ECOG) Performance Status Scale of 0-2.

20. The CD47-SIRPα blocking agent for use according to claim 1, wherein the human subject meets at least one of the criteria a) and b):
a) bone marrow assessment characterized by:
i. WBC<20,000/μL, wherein hydroxyurea can be given to bring WBC count to <20,000/μl;
ii. Platelet count≥50,000/μL without transfusion support; and/or
iii. Hemoglobin≥9 g/dL, wherein transfusion is allowed to achieve this Hb; and/or,
b) organ function characterized by:
i. Total Bilirubin≤1.5×Upper Limit Normal (ULN); or ≤2.5×ULN if the subject has Gilbert's syndrome;
ii. AST (SGOT)≤3×ULN (≤5×ULN if the subject has liver metastases);
iii. ALT (SGPT)≤3×ULN (≤5×ULN if the subject liver metastases);
iv. Creatinine clearance (CrCl)≥60 mL/min; and/or
v. Albumin≥3.0 g/dL.

21. The CD47-SIRPα blocking agent for use according to claim 1, wherein the CD47-SIRPα blocking agent and Azacitidine are administered simultaneously or sequentially.

22. The CD47-SIRPα blocking agent for use according to claim 1, wherein the CD47-SIRPα blocking agent is administered to the subject via an oral route.

23. The CD47-SIRPα blocking agent for use according to claim 1, wherein Azacitidine is administered to the subject via an oral route or a parenteral route, preferably via a subcutaneous injection or an intravenous infusion.

24. The CD47-SIRPα blocking agent for use according to claim 1, wherein the method comprises the administration of the compound of formula (I) at a total daily dose not exceeding 800 mg, preferably not exceeding 600 mg, more preferably not exceeding 500 mg, most preferably not exceeding 400 mg or the administration of a stereoisomer of the compound of formula (I) or a salt, solvate, amide or ester of the compound of formula (I) or stereoisomer thereof at the molar equivalent dose.

25. The CD47-SIRPα blocking agent for use according to claim 1, wherein the compound of formula (I) is administered to the subject once or twice daily.

26. The CD47-SIRPα blocking agent for use according to claim 1, wherein the method comprises the administration of the compound of formula (I) at a dose of 100 to 400 mg twice daily, preferably 100 mg, 200 mg, 300 mg, or 400 mg twice daily, or the twice daily administration of a stereoisomer of the compound of formula (I) or a salt, solvate, amide or ester of the compound of formula (I) or stereoisomer thereof at the molar equivalent dose.

27. The CD47-SIRPα blocking agent for use according to claim 1, wherein the method comprises the administration of the compound of formula (I) at a dose of 200 to 800 mg once daily, preferably 200 mg, 400 mg, 600 mg, or 800 mg once daily, or the once daily administration of a stereoisomer of the compound of formula (I) or a salt, solvate, amide or ester of the compound of formula (I) or stereoisomer thereof at the molar equivalent dose.

28. The CD47-SIRPα blocking agent for use according to claim 1, wherein the method further comprises the step of modifying the treatment in case the subject shows sign of prohibitive toxicity and/or disease progression.

29. The CD47-SIRPα blocking agent for use according to claim 28, wherein the step of modifying the treatment comprises:
i. a reduction in the total daily dose and/or dosing frequency, wherein the treatment is continued at a relatively lower daily dose and/or a reduced dosing frequency of the compound of formula (I);
ii. a dose hold, wherein the treatment is interrupted until the subject displays an improvement in prohibitive toxicity, and then reinitiating the treatment with the same daily dose and/or dosing frequency of the compound of formula (I); or
iii. a schedule change, wherein the treatment is interrupted until the subject displays improvement in prohibitive toxicity, and then reinitiating the treatment with a relatively lower daily dose and/or a reduced dosing frequency of the compound of formula (I).

30. The CD47-SIRPα blocking agent for use according to claim 28, wherein the prohibitive toxicity is a dose limiting toxicity (DLT).
